(19) United States
(12) Patent Application Publication
(10) Pub. No.: US 2024/0233898 A9
(43) Pub. Date: Jul. 11, 2024

(54) METHOD AND SYSTEM FOR PROVIDING MEDICAL SERVICE FOR CLINICAL DIAGNOSIS AND PRESCRIPTION

(71) Applicant: AIOBIO CORP., Seoul (KR)

(72) Inventor: Hong Cheol Yoon, Seoul (KR)

(21) Appl. No.: 18/511,939

(22) Filed: Nov. 16, 2023

Related U.S. Application Data
(63) Continuation of application No. PCT/KR2022/007192, filed on May 19, 2022.

(30) Foreign Application Priority Data
May 20, 2021 (KR) 10-2021-0064533
Sep. 2, 2021 (KR) 10-2021-0117051
Feb. 22, 2022 (KR) 10-2022-0023169
Apr. 11, 2022 (KR) 10-2022-0044731

(51) Int. Cl. G16H 20/00; G16H 10/60; G16H 15/00; G16H 50/20

(52) U.S. Cl. CPC G16H 20/00; G16H 10/60; G16H 15/00; G16H 50/20

(57) ABSTRACT

The present disclosure relates to a method of providing a medical service for clinical diagnosis and prescription, and a system therefor. More particularly, the present disclosure relates to a service providing method and system that generates dental data codes based on teeth information photographed from an examination device, generates and provides clinical diagnosis support data or prescription information required for prescription using the teeth information and dental data codes to a diagnostician or user, and furthermore collects prescription execution information from a device, which is used by the user to manage his or her teeth, to generate and provide feedback information on whether an appropriate prescription action or treatment action has been carried out based thereon, thereby aiding the user in maintaining and managing dental health.

### CROSS-REFERENCE TO RELATED APPLICATIONS

This application is a Continuation of Application No. PCT/KR2022/007192, filed on May 19, 2022, which in turn claims the benefit of Korean Patent Applications, No. 10-2021-0064533, filed on May 20, 2021, No. 10-2021-0117051, filed on Sep. 2, 2021, No. 10-2022-0023169, filed on Feb. 22, 2022, and No. 10-2022-0044731, filed on Apr. 11, 2022. The entire disclosures of all these applications are hereby incorporated by reference.

### TECHNICAL FIELD

The present disclosure relates to a method of providing a medical service for clinical diagnosis and prescription, and a system therefor. More particularly, the present disclosure relates to a service providing method and system that generates dental data codes based on teeth information photographed from an examination device, generates and provides clinical diagnosis support data or prescription information required for prescription using the teeth information and dental data codes to a diagnostician or user, and furthermore collects prescription execution information from a device, which is used by the user to manage his or her teeth, to generate and provide feedback information on whether an appropriate prescription action or treatment action has been carried out based thereon, thereby aiding the user in maintaining and managing dental health.

### BACKGROUND ART

The era of digital health care has arrived where information on personal health and medical care can be easily acquired through smart devices, systems, and platforms.

Digital health care, as the term suggests, means the transition of medical and health care services to a digital environment, wherein the core technologies of digital transformation, such as IoT (Internet of Things), cloud computing, sensors, big data, and artificial intelligence (AI) are combined with health care.

With the advent of this digital health care era, attempts are also being made to convert services related to dental treatment to a digital environment and utilize them in a variety of ways.

For example, a dental care service in the digital health care era may include a process of acquiring teeth information including a patient's teeth image through an oral scanner (photographing apparatus), a process of outputting this teeth information to a doctor's or patient's smart device, a process of receiving, by a treatment system capable of AI learning, the teeth information to output treatment results corresponding thereto, and the like.

Meanwhile, despite the trend of the times, digital health care technologies have not yet fully penetrated the medical field, especially the dental field, and there are many cases where past treatment and prescription methods are still used.

However, such a conventional treatment and prescription may be often carried out based on insufficient or inaccurate information resulting from checking teeth conditions only with the naked eye, and accordingly, dentists may be placed in an environment where they have no choice but to provide a treatment and prescription relying on professional knowledge rather than objective data, which as a result, leads to problems that do not meet today's patients' expectations for treatment or prescription quality. Furthermore, the effort or time required for treatment and prescriptions is not reduced at all, which may cause problems such as inconvenience to both patients and doctors.

On the other hand, in recent years, various devices and applications are being released and commercialized so that anyone can take care of their teeth at home, and however, in producing these devices and applications to provide services, it is important to implement an environment that makes it easy for general users without professional knowledge in the dental field to use. However, there is still a lack of channels for general users to obtain professional dental care methods other than Internet searches, especially dental care methods that suit them, and the only way to take care of their teeth is by relying on superficial search information.

The present disclosure is derived from the foregoing problems to solve the technical problems as mentioned above and to provide additional technical elements that cannot be easily invented by those skilled in the art.

Additionally, for reference, the present disclosure was supported by the following national research and development projects.

[Project Identification Number] 1465033392

[Project Number] HI20C0129010021

[Name of Ministry] Ministry of Health and Welfare

[Name of Project Management (Specialized) Organization] Korea Health Industry Development Institute

[Name of Research Program] Medical Technology Research and Development (R&D) in Dentistry

[Title of Research Project] Development of Multi-functional Smart Probe for Oral Disease Evaluation and Clinical Decision Support System Based on Convergence Data

[Contribution Rate] 1/1

[Project Performing Organization] AioBio Co., Ltd. [Research Period] Apr. 23, 2020 to Dec. 31, 2023

[Project Identification Number] 1711129562

[Project Number] 2018M3A9F102502621

[Name of Ministry] Ministry of Science and ICT

[Name of Project Management (Specialized) Organization] National Research Foundation of Korea

[Name of Research Program] Bio and Medical Technology Development (R&D)

[Title of Research Project] Development of Oral Biofilm-Based Mobile Healthcare Technology [Contribution Rate] 1/1

[Project Performing Organization] AioBio Co., Ltd.

[Research Period] Jan. 1, 2021 to Dec. 31, 2021

### DISCLOSURE OF INVENTION

### Technical Problem

In order to solve a technical problem, the present disclosure aims to define a data set that can be called dental data codes to acquire teeth information or process the acquired information, and to allow a computing apparatus to generate the above dental data codes based on photographed teeth images, thereby enabling them to be utilized in various areas.

Furthermore, the present disclosure also aims to generate images so as to see all teeth at a glance using dental data codes, thereby improving users' convenience in various areas.

In order to solve another technical problem, the present disclosure aims to easily identify information on observation items, inspection items, teeth condition values, evaluation values, or a degree of progress of a procedure from dental data codes, or allow a computing apparatus to easily recognize them, thereby obtaining comprehensive information on teeth only with simple codes.

The present disclosure aims to provide a system that analyzes the acquired teeth information (or examination information) and generates clinical diagnosis support data with reference to a previously stored cumulative database.

Furthermore, the present disclosure aims to allow a diagnostician to arbitrarily set up a diagnosis support algorithm so as to receive his or her desired clinical diagnosis support data.

In addition, the present disclosure aims to store clinical diagnosis support data provided by a plurality of diagnosticians on the system, build big data, and identify tendencies in diagnoses made by a plurality of diagnosticians according to a patient's gender, age, and diagnosis period and provide the relevant information to the diagnosticians.

Furthermore, the present disclosure aims to provide a system that generates prescription information based on the acquired teeth information (or examination information).

Furthermore, the present disclosure aims to provide users' devices in connection with a prescription system to allow the sharing and utilization of prescription information, or applications installed on the devices.

Furthermore, the present disclosure aims to generate and provide feedback information calculated based on the acquired teeth information or prescription information, and prescription execution information collected from the devices (information on dental care actions performed by an actual user).

Technical problems of the present disclosure are not limited to the above-mentioned problems, and other technical problems which are not mentioned herein will be clearly understood by those skilled in the art from the description below.

### Technical Solution

In order to solve the foregoing problems, a method of providing a medical service according to the present disclosure relates to a method of providing, by a teeth information processing system included in a treatment system, a medical service through processing teeth information and utilizing the processed information, and the method may include generating a dental data code for at least one tooth in a teeth image acquired by a computing device in the teeth information processing system, wherein the dental data code includes a coordinate code for identifying an intraoral location of at least each tooth.

Furthermore, in the medical service provision method, the dental data code may further include an inspection code for distinguishing a type of teeth inspection item; and an evaluation code indicating a condition according to an inspection result of teeth that have been inspected.

Furthermore, the medical service provision method may further include acquiring a photographed image of teeth, prior to generating the dental data code; acquiring a teeth development image based on the photographed image; and identifying individual tooth surfaces from the teeth development image.

Meanwhile, a method of providing a medical service according to another embodiment of the present disclosure relates to a method of providing, by a diagnosis support system included in a treatment system, a medical service for diagnosis support to a certain person, and the method may include receiving an input from a diagnostician terminal to define a plurality of factors and a diagnosis support algorithm; acquiring examination information, the examination information being information on the shape, location or condition of a patient's teeth, from an examination device; executing the defined diagnosis support algorithm to generate diagnosis support data; and providing the diagnosis support data to the diagnostician terminal.

Furthermore, in the medical service provision method, the executing of the diagnosis support algorithm may include preprocessing the examination information; standardizing the examination information; and generating diagnosis support data based on the standardized examination information.

Furthermore, in the medical service provision method, the preprocessing of the examination information may include analyzing the examination information; removing the unnecessary data; and determining whether the minimum examination information required to generate the diagnosis support data has been acquired.

Meanwhile, a method of providing a medical service according to still another embodiment of the present disclosure relates to method of providing, by a prescription system included in a treatment system, a medical service for a prescription to a certain diagnostician or user, and the method may include acquiring teeth information related to the teeth of a certain subject person; and performing calculation based on the teeth information, and generating prescription information corresponding to the subject person, wherein the teeth information includes at least one of the location information of the teeth and the condition information of the teeth.

Furthermore, the medical service provision method may further include, subsequent to generating the prescription information, transmitting the generated prescription information to a certain device, the device including a dental care device.

Furthermore, the medical service provision method may further include, subsequent to transmitting the generated prescription information to a certain device, receiving prescription execution information from the device, wherein the prescription execution information includes a value, an image, a video, or a code readable by the prescription system according to a user's prescription execution action.

Furthermore, the medical service provision method may further include, subsequent to receiving prescription execution information from the device, performing calculation based on either one of the teeth information and the prescription information, and the prescription execution information to generate feedback information.

### Advantageous Effects

According to the present disclosure as described above, dental data codes may be generated from images that have photographed teeth, thereby having an effect of allowing a user or a computing apparatus to identify comprehensive information on all teeth at a glance, and accurately.

Furthermore, the computing apparatus may be allowed to handle only text (numerals and/or letters)-based data or two-dimensional images that are relatively easy to calculate, thereby having an effect of significantly reducing resources required for calculation.

In addition, according to the present disclosure, data consisting only of two-dimensional images or simple text codes (dental data codes) may be utilized, thereby having an effect of easily transferring data that must be transmitted and received between terminals, for example, between a smartphone and a service server, to provide a certain service.

Moreover, according to the present disclosure, examination information acquired by a diagnosis support system may be analyzed and clinical diagnosis support data may be generated with reference to a previously stored cumulative database and provided to a diagnostician, thereby having an effect of carrying out more accurate diagnosis of patients.

Besides, according to the present disclosure, information that can be used as a guide or instruction may be provided to diagnosticians who lack experience and knowledge or who lack confidence in diagnosis, thereby providing confidence in the diagnostic action, as well as having an effect of lowering the overall possibility of misdiagnosis.

Furthermore, according to the present disclosure, a diagnostician may arbitrarily set up a diagnosis support algorithm to allow the diagnostician to receive his or her desired clinical diagnosis support data, and to have his or her own diagnostic algorithm or diagnostic routine, thereby having an effect of improving the outcome and efficiency of a diagnostic process.

In addition, according to the present disclosure, the diagnosis support system may provide diagnosticians with information on various options and tendencies related to diagnosis, thereby having an effect of preventing diagnostic errors and improving the accuracy of diagnosis.

Moreover, according to the present disclosure, a prescription system may generate prescription information based on the acquired teeth information and provide the prescription information, thereby having an effect of saving time required for treatment as well as providing accurate and systematic prescriptions.

Besides, according to the present disclosure, the prescription system may generate and provide feedback information through calculation based on the acquired teeth information and information on the user's action from a device so as to aid the user in taking care of his or her teeth, as well as complement insufficient dental care methods and obtain information on dental diseases that have been poorly understood, thereby having an effect of ultimately improving the user's dental health.

The effects of the present disclosure are not limited to the above-mentioned effects, and other effects that are not mentioned herein will be clearly understood by those skilled in the art from the description below.

### BRIEF DESCRIPTION OF THE DRAWINGS

FIG. 1 is a diagram schematically showing a process of providing a medical service for clinical diagnosis and prescription according to the present disclosure.

FIG. 2 is a diagram for understanding the concept of teeth information processing according to the present disclosure.

FIG. 3A and FIG. 3B are a diagram for explaining dental data codes (DDCs) and a dental data code set that is a set thereof.

FIG. 4 is a diagram showing the coordinate codes of respective teeth written in a two-dimensional standard image.

FIG. 5 is a diagram showing a dental data code according to another embodiment of the present disclosure.

FIG. 6A and FIG. 6B are a diagram showing an embodiment in which one additional digit is added to the coordinate code.

FIG. 7 shows an example of a two-dimensional standard image that can be generated when using the coordinate code according to FIG. 5.

FIGS. 8 and 9 are diagrams specifically showing a teeth information processing method.

FIG. 10 is a diagram showing a shape in which a diagnosis support system according to the present disclosure provides diagnosis support data to a diagnostician.

FIG. 11 is a diagram showing a process of generating diagnosis support data through a simple image.

FIG. 12 is a diagram showing a form in which a risk index and a risk group are calculated by weighting some of parameters included in a diagnosis support algorithm.

FIG. 13 is a diagram specifically showing a method of providing a medical service according to a first embodiment of the present disclosure.

FIG. 14 is a diagram specifically showing a method of providing a medical service according to a second embodiment of the present disclosure.

FIG. 15 is a diagram specifically showing a method of providing a medical service according to a third embodiment of the present disclosure.

FIG. 16 is a diagram showing a process of providing, by a diagnosis support system according to the present disclosure, a diagnosis tendency to a diagnostician.

FIG. 17 is a diagram specifically showing a method of providing a medical service according to a fourth embodiment of the present disclosure.

FIG. 18 is a diagram showing in detail a step of preprocessing examination information in the method of providing a medical service according to the fourth embodiment of the present disclosure.

FIG. 19 is a diagram showing a process of removing, by the diagnosis support system according to the present disclosure, unnecessary data and extracting only necessary data.

FIG. 20 is a diagram showing dental data codes used in the diagnosis support system.

FIG. 21 is a diagram showing a conventional functional teeth lifespan curve.

FIG. 22 is a diagram showing diagnosis support data including the functional teeth lifespan curve of the present disclosure.

FIG. 23 is a diagram showing a shape in which sales-related information is included in the diagnosis support data of the present disclosure.

FIG. 24 is a diagram showing a shape in which another sales-related information is included in the diagnosis support data of the present disclosure.

FIG. 25 is a diagram showing a shape in which information on future profits is included in the diagnosis support data of the present disclosure.

FIG. 26 is a diagram specifically showing a method of providing a medical service according to a seventh embodiment of the present disclosure.

FIG. 27 is a diagram specifically showing a method of providing a medical service according to an eighth embodiment of the present disclosure.

FIGS. 28A to 28D are diagrams showing various types of user devices and configurations thereof.

FIG. 29 is a diagram specifically showing a method of providing a medical service according to a ninth embodiment of the present disclosure.

FIG. 30 is a diagram specifically showing a method of providing a medical service according to a tenth embodiment of the present disclosure.

FIG. 31 is a diagram specifically showing a method of providing a medical service according to an eleventh embodiment of the present disclosure.

FIG. 32 is a diagram specifically showing a method of providing a medical service according to a twelfth embodiment of the present disclosure.

### DESCRIPTION OF SYMBOLS

10: Examination device 20: Diagnostician terminal 20B: Another diagnostician terminal
30: User terminal 40: User device 41, 51: Sensing unit
41a: 3D acceleration sensor 41b: 3D gyro sensor 41c: Geomagnetic field sensor
42, 52: Computing unit 43, 53: Communication unit 43a: Bluetooth communication unit
44, 54: Notification unit 45: Power supply unit 50: Ring-type device
60: Band-type device 100: Treatment system
110: Teeth information processing system
120: Diagnosis support system
130: Prescription System

### BEST MODE FOR CARRYING OUT THE INVENTION

Hereinafter, preferred embodiments of the present disclosure will be described in detail with reference to the accompanying drawings. Advantages and features of the present disclosure, and methods of accomplishing the same will be clearly understood with reference to the following embodiments described in detail in conjunction with the accompanying drawings. However, the present disclosure is not limited to those embodiments disclosed below but may be implemented in various different forms. It should be noted that the present embodiments are merely provided to make a full disclosure of the invention and also to allow those skilled in the art to know the full range of the invention, and therefore, the present disclosure is to be defined only by the scope of the appended claims. Further, like reference numerals refer to like or similar elements throughout the specification.

Unless otherwise defined, all terms (including technical and scientific terms) used in this specification may be used with meanings that can be commonly understood by those skilled in the art to which the present disclosure pertains. Additionally, terms defined in commonly used dictionaries are not interpreted ideally or excessively unless clearly specifically defined. It should be noted that the terms used herein are merely used to describe the embodiments, but not to limit the present disclosure. In this specification, unless clearly used otherwise, expressions in a singular form include a plural form.

Terms such as “first” and “second” are used to distinguish one element from another element, and the scope of rights should not be limited by those terms. For example, a first element may be termed as a second element, and likewise, a second element may be termed as a first element.

The term “comprises” and/or “comprising” used in the specification intend to express a constituent element, a step, an operation and/or a device does not exclude the existence or addition of one or more other constituent elements, steps, operations and/or devices.

A system of providing a medical service for clinical diagnosis and prescription (hereinafter, abbreviated as a treatment system 100) according to the present disclosure includes a teeth information processing system 110, a diagnosis support system 120, and a prescription system 130.

The treatment system 100 of the present disclosure provides a medical service to a diagnostician or a patient through three major steps. Specifically, a first step is generating and processing, by the teeth information processing system 110, dental data codes (DDCs) based on teeth information acquired through the examination device 10 (\textcircled{1}), a second step is analyzing, by the diagnosis support system 120, the teeth information and dental data codes to provide a clinical diagnosis support service (\textcircled{2}), and a third step is providing, by the prescription system 130, prescription information to a diagnostician or user based on a result value of the analyzed teeth information and dental data codes (\textcircled{3}).

The foregoing step of providing a medical service will be described step by step in sequence, and first, a method of generating and processing teeth information, which is a first step, will be described.

<Method of Generating and Processing Teeth Information (\textcircled{1})>

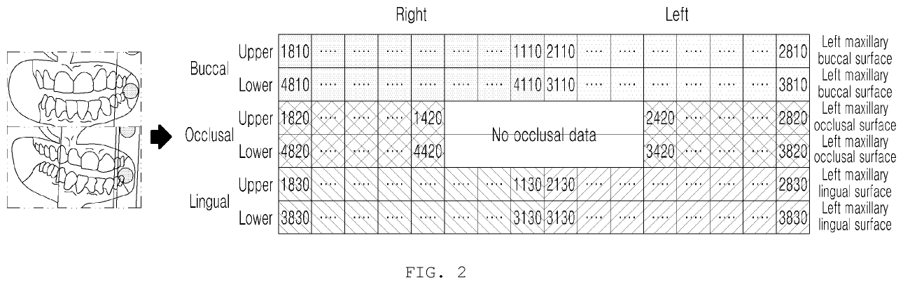

FIGS. 2 to 9 are diagrams for explaining a teeth information generation and processing method (\textcircled{1}).

First, FIG. 2 shows a shape of a teeth image photographed to facilitate the general understanding of the teeth information generation and processing method (\textcircled{1}) of the present disclosure, and a dental data code (DDC) image converted therefrom or generated based thereon. Referring to FIG. 2, a resultant output of photographing a certain patient's teeth or teeth structure using a camera is shown on the left side, and a dental data code image in which visible faces of respective teeth are arranged on a plane to allow the shape of the entire teeth to be seen at a glance is shown on the right side.

For reference, the dental data code image shown on the right side of FIG. 2 may be generated and provided to allow the user to easily recognize the overall condition of the teeth, and respective cells may correspond to the faces of observable teeth, and dental data codes may be written inside the respective cells. In addition, as will be described later, the respective cells may be internally divided again into sections, and respective sections may be made to correspond to the faces of an arbitrary tooth.

Meanwhile, the teeth image shown on the left side of FIG. 2 shows a resultant output subsequent to photographing an inside of the patient's oral cavity with an arbitrary examination device 10 (e.g., photographing apparatus (camera)), and these resultant outputs may be connected to one another in a continuous manner using image stitching technology, or the like, and this type of image is called a teeth development image. In this way, the patient's teeth may be photographed by the examination device from various directions, and a plurality of images or one or more videos obtained as resultant outputs of the photographing may be utilized to generate a teeth development image. As mentioned above, image stitching technology may be applied to multiple images to be stitched together on a plane to generate a teeth development image, and for videos, multiple images photographed by a video camera may be stitched together to generate a teeth development image.

[Definition and Structure of Dental Data Code]

A Dental Data Code (DDC) refers to information on the condition of an individual tooth expressed as a series of numerals, letters, or a combination of numerals and letters. Basically, there may be more than the number of teeth present in an oral cavity, and when dental data codes are assigned separately to the faces of respective teeth, there may exist dental data codes by matching the number of faces of the teeth.

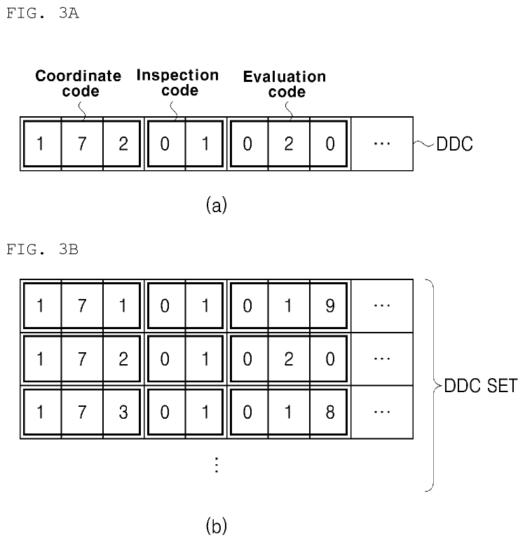

FIG. 3 shows an example of a dental data code to be defined in the present disclosure, wherein it can be seen that the dental data code of FIG. 3 consists only of a combination of numerals. In addition, when referring to FIG. 3, it can be seen that the dental data code mentioned in this detailed description includes at least one of a coordinate code representing the coordinates of a tooth, an inspection code representing an examination item for a tooth, and an evaluation code representing a result of examining each tooth.

Hereinafter, specific codes that make up the dental data code will be described in more detail.

First, the coordinate code may be a code for identifying each tooth present in an oral cavity, or furthermore, a code for identifying an observation surface on which each tooth is observed. For reference, in the dental field, each tooth is classified by location using a notation called Federation Dentaire International (FDI), and the coordinate code in the present disclosure partially refers to the above FDI, but differs from a conventional FDI notation in that the code further includes numerals (or letters) for identifying the observation surfaces of respective teeth.

The coordinate code in the present disclosure may consist of a 3-digit number as shown in FIG. 3, of which the first two digits may indicate a location of the tooth in the oral cavity, and the third numeral may indicate a direction of the observation surface. For example, a numeral written in the tens place of the first two digits is to indicate the left, right, top and bottom of the patient's teeth, wherein numerals “1”, “2”, “3” and “4” indicate the upper right, the upper left, the lower left, and the lower right of the patient's teeth, respectively. In addition, the number written in the ones place of the first two digits is a numeral to distinguish the order of each tooth in a dentition, wherein a first incisor (central incisor) corresponding to the middle of the dentition, a second incisor (lateral incisor), a canine (eyetooth), two premolars, two molars, and the last wisdom tooth may be shown as numerals, “1”, “2”, “3”, “4” (first premolar), “5” (second premolar), “6” (first molar), “7” (second molar” and “8”, respectively.

Furthermore, referring to FIG. 3, the third numeral of the coordinate code is to indicate a direction of the tooth observation surface, wherein, for example, from the patient's perspective, a buccal surface, an occlusal surface, a lingual surface may be shown as numerals, “1”, “2”, and “3”, respectively.

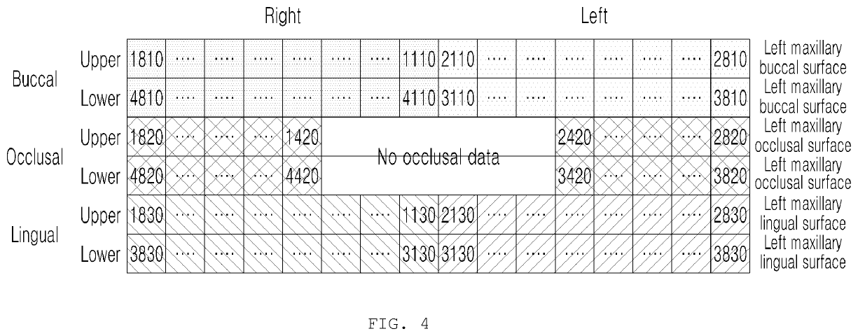

In this way, the coordinate code in the present disclosure may indicate a location of the tooth in the oral cavity and an observation surface to be viewed by utilizing three numerals. For reference, FIG. 4 shows the upper/lower left/right occlusal surfaces, upper/lower left/right buccal surfaces, upper/lower left/right lingual surfaces, anterior lingual surfaces, and anterior buccal surfaces of respective teeth in the oral cavity as a two-dimensional image, and it can be seen that coordinate codes are assigned to the first three digits. (The dental data code in FIG. 4 is shown as a total of 4 digits, but herein, will be described, exclusive of the numeral “0” written in the ones place) For an example, in consideration of which tooth a coordinate code ‘181’ refers to, the left two-digit number ‘18’ represents a third molar (wisdom tooth) of the right maxilla, and the remaining one-digit number ‘1’ represents a buccal surface. Therefore, the coordinate code of ‘181’ may be specified as a buccal surface of the third molar (wisdom tooth) located in the right maxilla. Next, the inspection code will be described. The inspection code, which is a code for distinguishing a type of teeth inspection or inspection item, and for example, when inspecting the temperature, volume, plaque, cavities, or wear of each tooth, a different number may be assigned to each inspection item. However, the inspection code may not be necessarily limited to distinguish the type of teeth inspection, and may also be utilized to distinguish the type of teeth inspection equipment. For example, assuming that an apparatus for inspecting cavities is separately present, a predefined number may be assigned to a result acquired using the apparatus, or assuming that an apparatus for inspecting microbial activity on teeth, another predefined number may be assigned to a result acquired by using the apparatus.

Next, the evaluation code will be described. The evaluation code denotes that a condition of the inspected tooth according to the inspection result is shown as a number according to a degree thereof. For example, a tooth condition value for a severity of the tooth, a risk of the tooth, a priority of the procedure, and the like may be expressed as a 3-digit numeric value and included in the dental data code. More specifically, assuming that an inspection related to cracks present on a tooth surface has been carried out, the number 999 may be assigned to a tooth with the most severe degree of cracks, and the number 001 may be assigned to the tooth with the weakest degree of cracks. Furthermore, in this case, just because the evaluation code has three digits, it does not necessarily mean that all evaluation items must be shown as numbers from 0 to 999, and a range of numbers may be adjusted to be used as needed. For example, there may also be evaluation items using numbers from 001 to 005. In addition, to emphasize once again, the evaluation code is not necessarily limited to a three-digit number, and the number of digits may be adjusted as needed. This evaluation code may be assigned by a computing apparatus (not shown) within the teeth information processing system, or may be assigned based on an operator's (dentist, etc.) input through the computing apparatus. In the former case, an arbitrary algorithm may be executed by the teeth information processing system to determine how severe the tooth is by analyzing an image of the photographed teeth.

Meanwhile, in addition to the coordinate code, inspection code, and evaluation code described above, a code for a procedure progress situation of the tooth may be further included. In this detailed description, this will be referred to as a procedure progress code. The procedure progress code may be a numerical representation of the procedure progress situation of a specific tooth in a dental data code, for example, the number of times the patient has visited a dental hospital, the number of procedures the patient has currently received among a plurality of procedures, or a sequence of the most recent procedure the patient has received among a series of procedures may be shown as numbers.

For reference, (a) of FIG. 3 shows a dental data code (DDC) that shows the location and condition of an individual tooth, and (b) of FIG. 3 shows a dental data code set containing a plurality of dental data codes. In other words, a dental data code set may be understood as a set of dental data codes for all teeth in a certain patient's oral cavity. As will be described later, this dental data code set may be used to implement a two-dimensional image, or a dental data code set may be generated from a two-dimensional image.

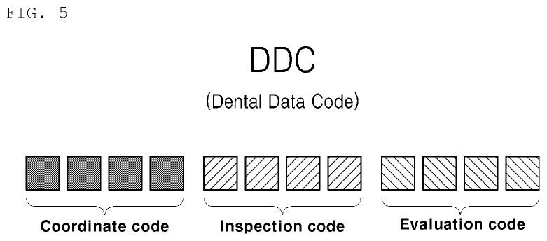

Meanwhile, FIG. 5 shows another embodiment of a dental data code generated by the teeth information processing method according to the present disclosure, wherein the dental data code in FIG. 5 consists of a total of 13 digits, and among them, the first 4-digit number represents the coordinate code, the next 4-digit number represents the inspection code, and the last 5-digit number represents the evaluation code. However, as mentioned above, the number of digits included in each code may vary, and it is to be understood that the dental data code shown in FIG. 5 is an example to aid the understanding of the present disclosure.

Compared to the dental data code described in FIG. 3, the dental data code in FIG. 5 is different in that the coordinate code consists of a total of 4 digits by adding one digit, and the inspection code is also different in that two digits are added, and particularly between them, functional effects and usability effects that can be achieved by extending the coordinate code to 4 digits will be further described with reference to FIGS. 5 to 7.

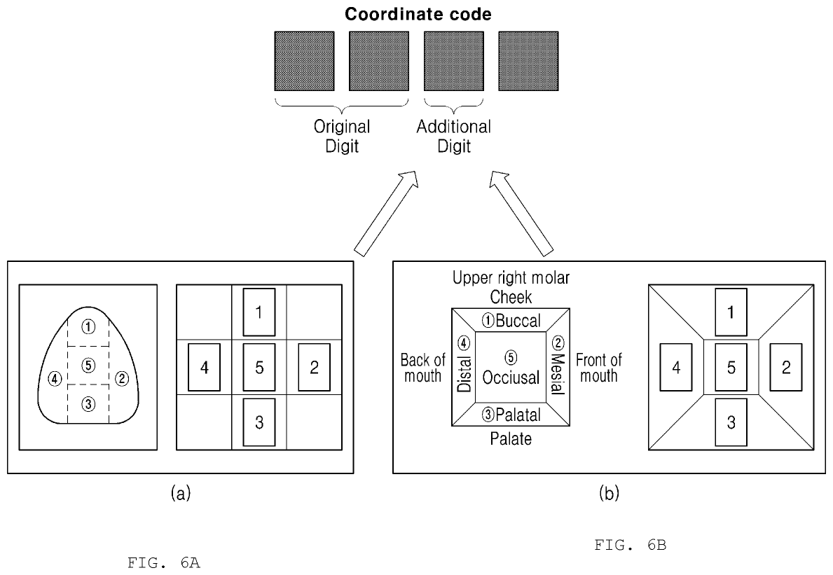

FIG. 6 shows a coordinate code with a total of 4 digits, which is extended by adding a single digit (additional digit) to the above-described 3-digit coordinate code (original digit), wherein the added single digit may be one for identifying a region on the tooth surface, or for identifying each face generated when the tooth is shaped into a polyhedron (preferably a square pyramid).

(a) of FIG. 6 shows an example in which one surface of a tooth is divided into five regions and each region is assigned a numeral from “1” to “5”. The left side of (a) of FIG. 6 shows a shape in which a tooth surface is divided into a total of five regions when looking at the front of a specific tooth, that is, an upper region (\textcircled{1}), a right region (\textcircled{2}), a lower region (\textcircled{3}), a left region (\textcircled{4}), a central region (\textcircled{5}), and the right side of (a) of FIG. 6 is a more simplified diagram thereof.

In (b) of FIG. 6, it is shown an example in which when one specific tooth that is an object of observation is shaped as a polyhedron (square pyramid), numerals from “1” to “5” are assigned to respective visible faces based on the plan view when the square pyramid is viewed from above. The left side of (b) of FIG. 6 shows a top view of the square pyramid when viewed from above, wherein it is shown a shape in which the square pyramid of the drawing is divided into an upper face (\textcircled{1}), a right face (\textcircled{2}), a lower face (\textcircled{3}), a left face (\textcircled{4}), a central face (\textcircled{5}), and the right side of (b) of FIG. 6 is a simplified diagram thereof. For reference, the square pyramid shown on the left side of (b) of FIG. 6 is shown on the assumption that the right side is a labial side, the left side is a lingual side, and the upper side is buccal side, but the assignment of directions thereof or digits according to the directions may be carried out in a different manner.

Meanwhile, to summarize the description of FIG. 6 again, the coordinate code may further include a digit to identify a specific region on a tooth surface defined in (a) or (b) of FIG. 6, or an individual surface on the tooth shaped as a polyhedron. In analyzing a patient's teeth, it is fundamental to observe an overall condition of the teeth, for example, whether there is plaque or cavities on the teeth, whether the teeth are fractured, and the like, and when observing the condition of teeth in this way, in case a problematic portion can be accurately identified and recorded, users (especially dentists) who will refer to the records may receive great aid in dental treatment or maintaining dental health. It is expected that the dental data code proposed in FIG. 6 above, especially the technical concept of adding additional information to the coordinate code, can be used to bring about this effect.

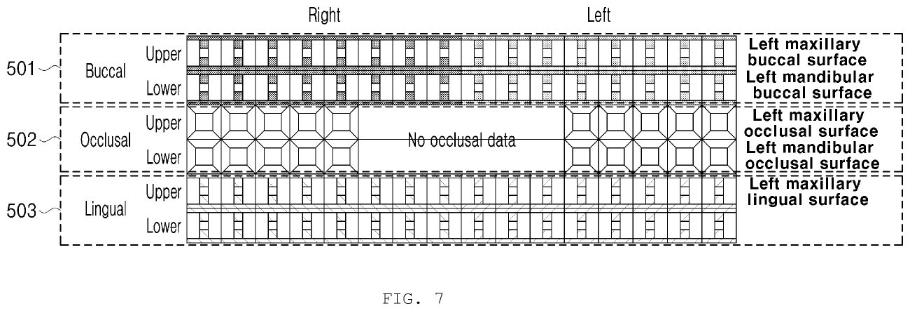

FIG. 7 shows an example of a two-dimensional standard image that can be generated when the coordinate code includes an additional digit as shown in FIG. 6. Referring to this drawing, the two-dimensional standard image is largely divided into a buccal view 501 showing a shape of the teeth as seen from a buccal side in the oral cavity, and an occlusal view 502 showing a shape of the teeth as seen from an occlusal side thereof, and a lingual view 503 showing a shape of the teeth as seen from a lingual side thereof. In addition, it can be seen that the buccal view 501 and the lingual view 503 show teeth in a state in which each region on the tooth surface is distinguished according to the method described in (a) of FIG. 6, and the occlusal view 502 shows teeth in a state in which each face on the square pyramid of the tooth is distinguished according to the method described in (b) of FIG. 6 in a diagrammatic manner. For reference, portions marked “no occlusal” in the drawing is a region corresponding to teeth with a small occlusal area value, and in general, an occlusal surface of teeth in this region does not have a disease alone, and often comes into contact with nearby biological tissues such as the buccal and lingual sides to cause a disease, and thus observation of the surrounding region is mainly carried out rather than the occlusal surface itself. FIG. 7 may also include a “no occlusal” region in a two-dimensional standard image in this context, but the embodiment is not necessarily limited to having the “no occlusal” region, and the occlusal surfaces of all teeth may be shown as needed.

In addition, as a matter of special note, the two-dimensional standard image in FIG. 7 has a different arrangement of occlusal surfaces in particular compared to the two-dimensional standard image in FIG. 4, wherein it is to be understood that not only the arrangement of occlusal surfaces but also an overall arrangement of observation surfaces may vary depending on the utilization of this two-dimensional standard image. For example, the arrangement may exist in various forms, such as a form that can be easily recognized by professionals such as dentists, or a form that can be easily recognized by the general public.

In addition, in FIG. 7, the dental data code is not separately shown within each cell or on sectional faces within each cell, but it has been described above that the foregoing data code can be generated within each cell, especially for each sectional faces within each cell, and thus it will be easily understood that the data code generated in this way can also be shown in an image such as in FIG. 7.

[Method of Generating Dental Data Code]

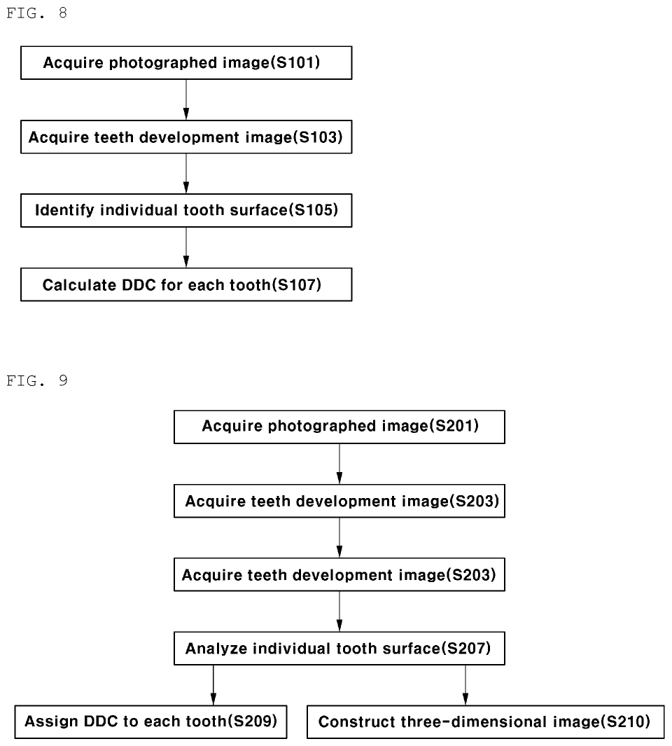

Next, a process of generating, by a computing apparatus (not shown) included in the teeth information processing system 110, a dental data code will be described. A dental data code may be generated by executing the so-called teeth information processing method, and representative examples thereof will be described through FIGS. 8 and 9.

Referring to FIG. 8, the computing apparatus may first perform a step (S101) of acquiring a photographed image of a tooth. A photographed image is a resultant output of an inside of a patient's oral cavity that can be obtained through a photographing apparatus. When an intraoral-only camera (scanner) or a photographing apparatus capable of photographing a certain object, even if it is not necessarily for oral cavity only, is inserted into the patient's oral cavity and then the teeth are photographed, images of the teeth taken from various directions or videos of the teeth taken continuously from various directions may be recorded, and the computing device may acquire the photographed images of the teeth in this way.

Next, the computing apparatus may perform a step (S103) of acquiring a teeth development image. In general, a development image refers to an image in which a surface of an object is unfolded on a plane, and therefore it will be understood that a teeth development image is an image in which the surfaces of respective teeth are unfolded on a plane from the photographed images. The computing apparatus may perform image stitching on previously photographed image(s), and based on the image stitching, multiple images may be connected to one another, and as a result, a teeth development image in the same form as a panoramic image may be acquired. Since image stitching technology has already been commercialized in the relevant fields, a detailed description thereof will be omitted in this detailed description.

Next, the computing apparatus may perform a step (S105) of identifying individual tooth surfaces through performing image segmentation on the previously acquired teeth development image. It will be understood that this step is a step of extracting only tooth surfaces from the teeth development image, and identifying each of the extracted tooth surface as a specific tooth, wherein for example, a process of extracting a front tooth surface from the teeth development image and then identifying the relevant tooth surface as a central incisor (front tooth) may be included in this step.

Image segmentation refers to a process of dividing a digital image into several sets of pixels, wherein image segmentation in the present disclosure may be used to find the boundaries (lines, curves) of respective tooth surfaces, especially in the teeth development image. Image segmentation may be carried out in various ways, and for example, a method using a threshold, an edge detection method, a region growth method, or a method using texture feature values may be used.

Meanwhile, after the tooth surface region is identified by image segmentation, the computing apparatus must identify which tooth the tooth surface is, wherein in this case, feature points of the identified tooth surface, a size of region on the identified tooth surface, or an appearance of the identified tooth surface may be referenced for the identification process. Furthermore, when any one tooth surface is identified as a specific tooth, the computing apparatus may identify tooth surfaces adjacent thereto using the specific tooth as a reference.

For reference, step S105 may further include a process of reconstructing the identified tooth surfaces into a two-dimensional standard image. That is, the identified tooth surfaces are displayed in a two-dimensional standard image consisting of a plurality of cells as seen in FIG. 4, and each tooth surface is shown in a corresponding cell, thereby checking the patient's all observable tooth surfaces at a glance.

An additional explanation regarding the two-dimensional standard image is as follows. The two-dimensional standard image does not take up much data space due to its smaller and lighter capacity compared to a three-dimensional teeth image, and thus has an advantage capable of storing a larger amount of teeth information in the same storage space. In addition, the two-dimensional standard image has an advantage of being faster when transmitting or receiving compared to a three-dimensional teeth image due to its small capacity. Additionally, the two-dimensional standard image has an advantage capable of determining the condition of respective teeth at a glance, such as plaque and cavities, compared to a three-dimensional teeth image.

Meanwhile, the two-dimensional standard image in the present disclosure has several features, one of which is that a root surface of each tooth is not included. This is because a surface corresponding to the root of the tooth cannot be scanned using a photographing apparatus with current technology, but in case a photographing apparatus capable of photographing the root side of the tooth is developed in the future, it is natural that the root side of the tooth will be arranged on the two-dimensional standard image.

In addition, an occlusal surface of the anterior maxilla and an occlusal surface of the anterior mandible are not shown in the two-dimensional standard image in the present disclosure, which is due to the fact that the operator can easily identify them with his or her naked eye even without necessarily converting them into image data, and that occlusal surfaces of the anterior maxilla and mandible are difficult to image because the occlusal areas thereof are relatively very small. However, similarly, the occlusal surfaces of the anterior maxilla and mandible may also be placed in the two-dimensional standard image as needed.

Meanwhile, subsequent to identifying the individual tooth surfaces as described above, the computing apparatus may calculate a dental data code for each tooth (S107). That is, in this step, the computing apparatus may perform assigning a dental data code to each identified tooth, and at this time, the dental data code may include the coordinate code, inspection code, and evaluation code as described above. The coordinate code may be determined when the tooth surface identified in the preceding step S105 is identified as a specific tooth, and the values of the inspection code and evaluation code may be determined based on the type of inspection (analysis) carried out on the identified tooth surface, the type of apparatus used to photograph the teeth, or the result values of the inspection (analysis).

For reference, in relation to the inspection code and evaluation code, the computing apparatus may determine the values of the inspection code and evaluation code with reference to oral examination data that is separately acquired and stored. The oral examination data means the results of inspecting teeth or tissues in the oral cavity converted into data, and this oral examination data may be in the form of image data. For example, when an inside of a patient's oral cavity is inspected using a biofilm fluorescence inspection (Qray inspection) apparatus that photographs a fluorescent image using a special filter, an image with a fluorescent color is photographed depending on a level of microbial activity on the tooth surface, and this image data may also be regarded as a type of oral examination data. Alternatively, the state of cavity formation or plaque formation may be acquired as image data by photographing an inside of the patient's oral cavity, which may also be regarded as a type of oral examination data.

Meanwhile, the oral examination data does not necessarily have to be in the form of image data, and the oral examination data may be text-type data consisting of numerals, letters, or a combination of numerals and letters. Additionally, when the oral examination data is in the form of image data, the oral examination data may be two-dimensional or three-dimensional image data.

The computing apparatus may refer to the oral examination data described above in assigning an inspection code and an evaluation code to the identified teeth, analyze the oral examination data according to predetermined criteria, calculate a value therefrom, and then use the calculated value as the inspection code and evaluation code.

The computing apparatus may calculate the dental data code for each tooth through steps S101 to S107 described above, and the dental data code calculated in this way may be used by itself, or as shown in FIG. 4, may also be constructed and utilized in the form of a two-dimensional standard image.

Meanwhile, FIG. 9 sequentially shows a method in which a computing device processes teeth information according to a second embodiment.

Referring to FIG. 9, steps S201 to S205 are substantially the same as those described in FIG. 8, and thus a detailed description thereof will be omitted herein.

Step S207 is analyzing individual tooth surfaces (S207), wherein the computing apparatus may analyze whether there is an abnormality with the individual tooth surfaces identified in the step S205. The analysis at this time may preferably involve image processing, but is not necessarily limited thereto.

Types of dental abnormalities that can be analyzed by step S207 may include damage caused by physical force applied to the tooth surface, such as crown residue, crown fracture, complex crown fracture, root fracture, and crown-root fracture, and diseases in which a tooth structure is destroyed due to bacterial causes such as cavities or plaque. For reference, in step S207, the Qray examination method briefly mentioned above in the description of FIG. 8 may also be used.

On the other hand, the computing apparatus may complete a dental data code set for all teeth by assigning a dental data code to each tooth (S209) after completing the analysis of individual tooth surfaces, or construct a three-dimensional image based on images that have identified the individual tooth surfaces or results that have analyzed the individual tooth surfaces (S210).

In step S209, dental data codes may be determined for all teeth, and the dental data codes determined in this way may be provided to another linked service group and used to provide a certain service.

Step S210 may be carried out by combining the identified individual tooth surfaces or images acquired from among the teeth development image with three-dimensional data separately acquired by the computing apparatus. To put it simply, the step may also be understood as a process of overlaying previously acquired tooth surfaces on the three-dimensional data, on the assumption that there is data that has previously been photographed an inside of the patient's oral cavity. In this case, the type of three-dimensional data may include X-ray data or CT data that has photographed the inside of the patient's oral cavity, and may include all of them if there is other data that can display the inside of the patient's oral cavity in a three-dimensional coordinate system.

When the computing apparatus merges individual tooth surface images into the three-dimensional data, the computing apparatus may compare three-dimensional data and a two-dimensional image for each individual tooth while determining a plurality of feature points, and searching for a shape in which these feature points match, thereby overlaying the two-dimensional image on the three-dimensional data. The three-dimensional image constructed in this way may be provided to a professional (dentist) or individual patient, and the three-dimensional image may be of great help to them in checking the condition of the patient's or his or her own teeth.

In the above, the teeth information generation and processing method (\textcircled{1}), which is a first step in the medical service of the present disclosure, has been described.

Next, the clinical diagnosis support provision service method (\textcircled{2}), which is a second step of the medical service of the present disclosure, will be described.

<Method of Providing Clinical Diagnosis Support Service (\textcircled{2})>

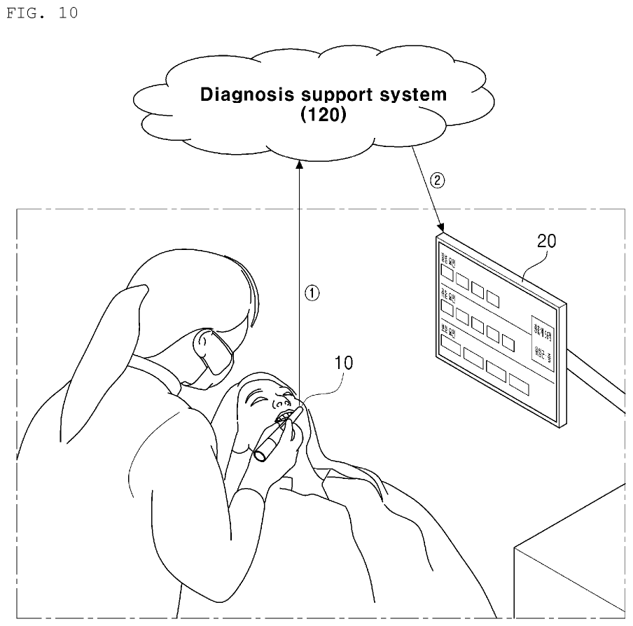

FIGS. 10 to 25 are diagrams for explaining the diagnosis support service provision method (\textcircled{3}).

in a treatment system according to the present disclosure, a clinical diagnosis support system (hereinafter abbreviated as diagnosis support system 120) is a system that provides information that can be obtained from patients in the dental field, and analysis resultant outputs that aid the diagnostician in making an accurate decision when performing a clinical diagnosis of the patient by referring to the previously stored cumulative database.

Prior to describing in detail the clinical diagnosis support provision service method of the present disclosure (\textcircled{2}), terms frequently mentioned in the process of providing the clinical diagnosis support service of the present disclosure will be described in brief.

FIG. 10 is a diagram showing a shape in which a diagnostician acquires information on a patient's teeth through the examination device 10 (FIG. 10-\textcircled{1}), and diagnosis support data is generated by referring to the patient's dental information acquired by the diagnosis support system 120 and the accumulated data already stored in the server's database, and the diagnosis support data is displayed through the diagnostician terminal 20 (FIG. 10-\textcircled{2}).

[Examination Device 10]

Referring to FIG. 10, the examination device 10 refers to a device used to acquire identifiable information from a patient, and there is no limit to the type of device as long as meaningful condition information can be obtained from the patient. For example, the examination device 10 may be an intraoral camera or a 3D intraoral scanner that photographs the patient's teeth as they are and displays the shape and location of the teeth in a two-dimensional or three-dimensional image, and may also be a quantitative light fluorescence apparatus that can obtain the detailed condition of teeth by irradiating light with a specific wavelength to determine a degree of caries (cavities) in the teeth. Moreover, in addition to the intraoral camera, 3D intraoral scanner, and quantitative light fluorescence apparatus described above, any device provided for acquisition of information that can be used as a basis for diagnosis before the diagnostician performs an accurate dental diagnosis for the patient, that is, information on the shape, location, or condition of the patient's teeth, may be included in the examination device 10 mentioned in this detailed description.

[Examination Information]

Information that can be used as a basis for diagnosis before the aforementioned diagnostician performs an accurate dental diagnosis for the patient, that is, information on the shape, location, or condition of the patient's teeth, may be expressed implicitly in the term examination information in the present disclosure. For reference, the examination information may include teeth information described above in the method of generating and processing teeth information (\textcircled{1}).

This examination information is used as a basic resource for the diagnostician to receive a clinical diagnosis support service according to the present disclosure. Specifically, the diagnostician may make his own diagnosis using only the examination information acquired through the examination device 10, but some diagnosticians who lack experience and knowledge may not be confident about the diagnosis and may have difficulties in making their own diagnosis, and the present disclosure may provide a clinical diagnosis support service to aid these diagnosticians in making an accurate diagnosis, and in order for the diagnosis support system 120 to provide a clinical diagnosis support service, it must receive examination information, which is reference data, and as a result, the examination information may become a basic resource for the diagnostician to receive the clinical diagnosis support service.

[Diagnosis Support System 120]

Referring to FIG. 10, the diagnosis support system 120 is a system that receives examination information from the examination device 10 and generates diagnosis support data through the received examination information and previously stored cumulative data (big data), and displays the generated diagnosis support data on the diagnostician terminal 20 so as to provide a clinical diagnosis support service to a diagnostician.

[Diagnostician Terminal 20]

The diagnostician terminal 20 described in the present disclosure may be any terminal that has computational functions and network functions, such as a desktop, a laptop, a smartphone, a tablet PC, a smart watch, and the like. In this detailed description, to aid the understanding of the disclosure, the diagnostic terminal 20 installed in a dental office as shown in FIG. 10 will be assumed as a representative diagnostic terminal 20.

[Diagnosis Support Algorithm and Diagnosis Support Data]

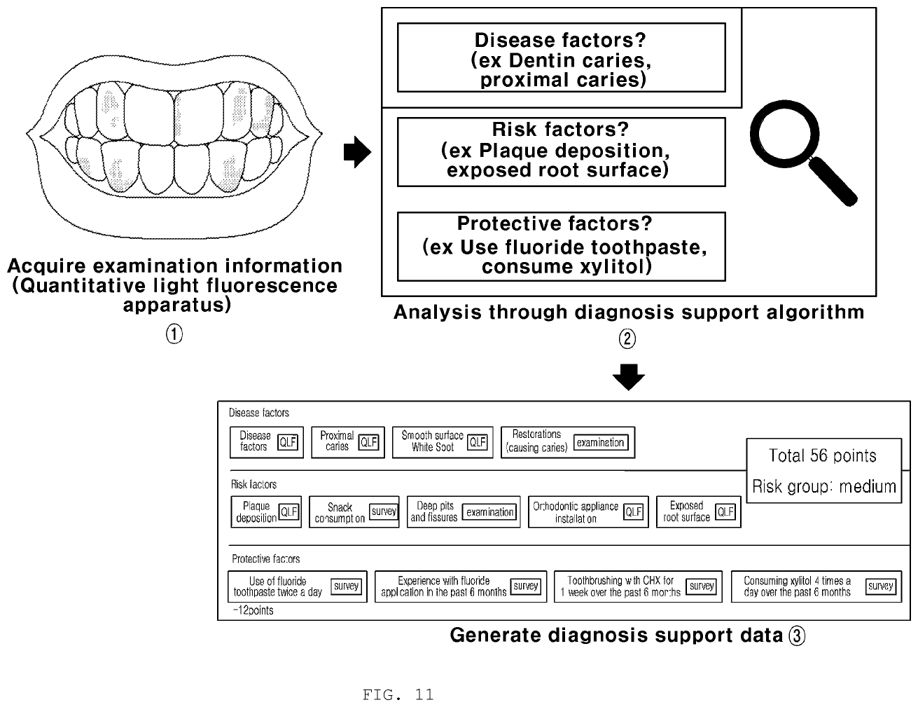

FIG. 11 is a diagram showing in brief a process of analyzing the examination information (\textcircled{1} in FIG. 11) acquired by the diagnosis support system 120 of the present disclosure using a preset diagnosis support algorithm (\textcircled{2} in FIG. 11) to generate diagnosis support data (\textcircled{3} in in FIG. 11).

The diagnosis support algorithm refers to a set of rules that generate diagnosis support data by analyzing examination information based on previously received setting values on the assumption that the diagnosis support system 120 has received setting values for several factors, such as disease factors, risk factors, or protective factors, from the diagnostician terminal 20. For reference, the disease factors may include the cause of a disease that can be identified from the examination information and the patient's teeth condition due to the disease, the risk factors may include the cause of triggering a disease identified from the examination information, and the protective factors may include factors that help improve the disease or factors that can prevent the disease.

Considering the process of generating diagnosis support data according to the diagnosis support algorithm based on these factors, first of all, the diagnostician may set the most necessary factors and/or diagnosis support algorithm to identify the patient's disease, and through the diagnosis support algorithm, factors set by the diagnostician may be analyzed and the resulting diagnosis support data may be provided to the diagnostician. For example, when the factors that the diagnostician wants to check while diagnosing a patient with severe toothache are ‘dentin caries’ and a ‘degree of proximal caries (confirmation of caries vulnerable area)’, the diagnostician may set ‘dentin caries’ and ‘proximal caries’ as disease factors through the diagnostician terminal 20 and even define a diagnosis support algorithm based on the disease factors, and furthermore, according to the diagnosis support algorithm, information on ‘dentin caries’ and ‘proximal caries’ may be analyzed from the examination information based on the above settings, and diagnostic support data, which is the analyzed result values, may be provided to the diagnostician. For reference, in the above embodiment, an example in which only ‘disease factors’ are used has been described for easy understanding of the disclosure but of course, it is to be understood that not only ‘disease factors’ but also ‘risk factors’ and ‘protective factors’ may also be used through the diagnosis support algorithm.

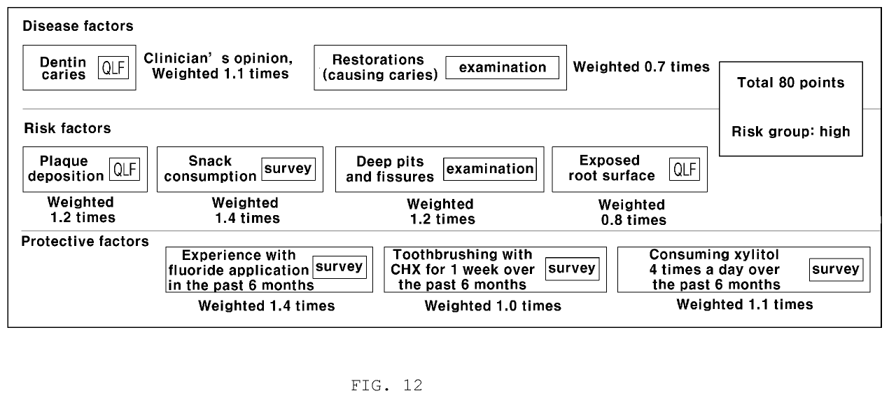

Meanwhile, the diagnosis support algorithm is designed to give weight to each of these factors, allowing the diagnostician to receive his or her desired information. Referring to FIG. 12, the weight may be an importance level given to individual factors when the diagnosis support algorithm calculates a risk index of the patient's teeth, and the greater the impact on dental health, the higher the weight, or the smaller the impact on dental health, the lower the weight may be assigned. These weight values may be set differently depending on the intention of each diagnostician. When the diagnostician assigns a weight to each of the factors included in the diagnosis support algorithm, the diagnosis support algorithm may generate diagnosis support data with the weights reflected, and the diagnostician may receive diagnosis support data in his or her desired manner or format.

[Method of Providing Clinical Diagnosis Support Service—\textcircled{1}: First Embodiment]

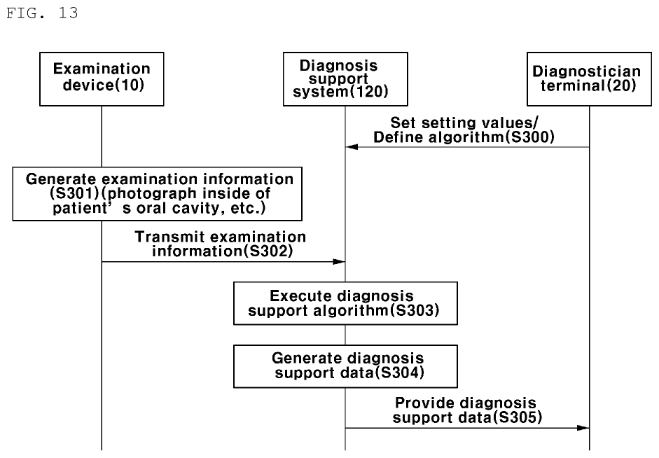

FIG. 13 is a diagram specifically showing a method of providing a clinical diagnosis support service according to a first embodiment of the present disclosure.

The method of providing a clinical diagnosis support service according to the first embodiment of the present disclosure may first start with accessing, by a diagnostician, the diagnosis support system 120 through his or her diagnostician terminal 20 and then determining factors (disease factors, risk factors, or protection factors, etc.) or defining a diagnosis support algorithm (S300). Simply put, this step may also be understood as a step in which a doctor customizes diagnosis support data he or she wants to receive when diagnosing a patient. That is, the doctor may set only the factors he or she wants to see from among various factors, and may also define an algorithm so as to receive his or her desired result values through a series of calculations from the set factors.

Subsequent to executing step S300, a step (S301) of generating examination information by the examination device 10 may be carried out. For example, a dentist who is a diagnostician may photograph the inside of a patient's oral cavity using the examination device 10, and accordingly, examination information such as an oral cavity image may be generated by the examination device 10.

Subsequent to generating the examination information, the examination device 10 transmits the generated examination information to the diagnosis support system 120 (S302). For reference, the examination information generated by the examination device 10 may be directly transmitted to the diagnosis support system 120 as described in step S302, but when the examination device 10 is not linked to the diagnosis support system 120 or the communication module is not provided in the examination device 10, the examination information acquired by the examination device 10 may be transmitted to the diagnosis support system 120 through the diagnostician terminal 20. For example, in an environment where an intraoral camera is connected to the diagnostician terminal 20, images photographed by the intraoral camera may be primarily stored inside the diagnostician terminal 20, and the stored images may be transmitted later to the diagnosis support system 120.

Subsequent to step S302, the diagnosis support algorithm may be executed by the diagnosis support system 120 (S303). The diagnosis support algorithm refers to an algorithm previously set or defined by the diagnostician in step S300 in the above, and the diagnosis support algorithm may be carried out based on the examination information received in step S302.

Subsequent to executing step S303, diagnosis support data is generated (S304). The diagnosis support data may exist in various formats, for example, in the form of images or videos, or in the form of numerical values or text.

Subsequent to generating the diagnosis support data, the diagnosis support system 120 provides the diagnosis support data to the diagnostician terminal 20 (S305), thereby completing the method of providing a clinical diagnosis support service according to the first embodiment of the present disclosure.

[Method of Providing Clinical Diagnosis Support Service—\textcircled{2}: Second Embodiment]

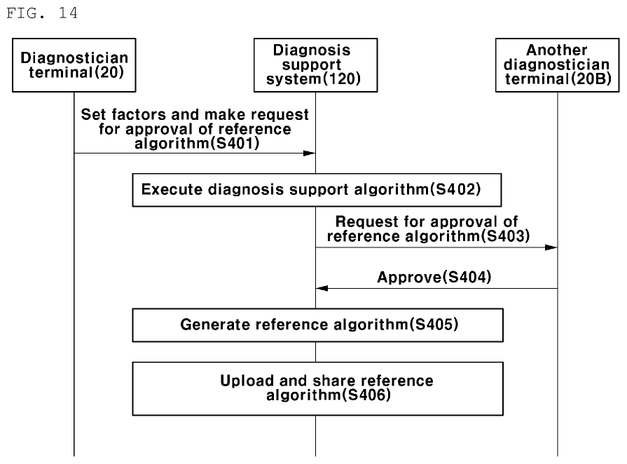

FIG. 14 is a diagram specifically showing a method of providing a clinical diagnosis service according to a second embodiment of the present disclosure.

The method of providing a clinical diagnosis support service according to the second embodiment of the present disclosure includes a process of generating and sharing a benchmark diagnosis support algorithm, that is, a reference algorithm, that can be provided to diagnosticians.

The reference algorithm may be an algorithm that can be provided to a large number of diagnosticians using the diagnosis support service according to the present disclosure, and since this reference algorithm is provided to a large number of diagnosticians, its reliability needs to be verified, and thus processes such as those in FIG. 14 may be required.

For example, when diagnosing children under the age of 7, a specific pediatric dentist may diagnose a specific disease based only on specific factors based on his or her own knowledge and experience, wherein the pediatric dentist may share his or her own factors and algorithm with other diagnosticians by registering them as a reference algorithm through the diagnosis support system 120.

In particular, in the case of dental hospitals, although there are specialized departments such as oral and maxillofacial surgery, dental prosthodontics, orthodontics, pediatric dentistry, periodontology, dental conservation, stomatology, oral and maxillofacial radiology, oral pathology, and preventive dentistry, the majority of private dentists do not operate dental hospitals exclusively for any one specialty, and therefore, depending on the patient, diagnostic know-how from a specific specialist may be required, and therefore, the diagnosis support system 120 according to the present disclosure may provide a reference algorithm to diagnosticians, thereby improving convenience for the diagnosticians.

In order to generate a reference algorithm, a step (S401) of setting and defining, by the diagnostician terminal 20, factors and an algorithm and making a request for approval of the reference algorithm from the diagnosis support system 120 may be carried out.

Then, the diagnosis support system 120 generates a diagnosis support algorithm based on the items set and defined by the diagnostician terminal 20 in step S401 (S402).

Subsequent to step S402, the diagnosis support system 120 makes a request for approval from other diagnostician terminals 20B as to whether the diagnosis support algorithm generated in step S402 can be used as a reference algorithm (S403). This process may be carried out on multiple other diagnosticians who have been designated as a professional pool in advance, and at this time, the multiple other diagnosticians who are the subjects of the approval request may be determined differently depending on the category, such as what disease the diagnosis support algorithm is for diagnosing, or what type of patient it is for diagnosing.

Subsequent to step S403, when the diagnosis support algorithm generated in step S402 is deemed appropriate as a reference algorithm, the other diagnostician terminal 20B approves it and transmits the approval to the diagnosis support system 120 (S404).

Subsequent to the approval, the diagnosis support system 120 recognizes and generates the diagnosis support algorithm in step S402 as a reference algorithm (S405), and uploads the generated reference algorithm into the server so as to be shared such that other diagnostic terminals 20B can receive the reference algorithm (S406).

[Method of Providing Clinical Diagnosis Support Service—\textcircled{3}: Third Embodiment]

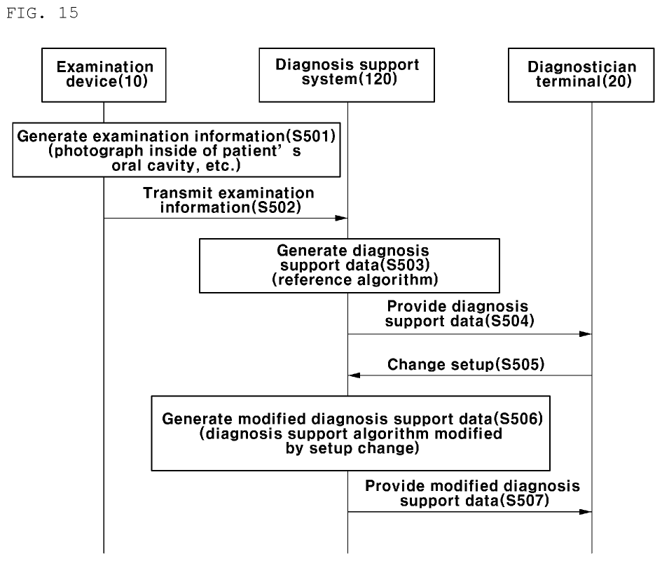

FIG. 15 is a diagram specifically showing a method of providing a clinical diagnosis support service according to a third embodiment of the present disclosure.

A method of providing a clinical diagnosis support service according to the third embodiment of the present disclosure includes a process of changing the settings when the diagnostician does not like the diagnosis support data generated by the reference algorithm or to generate his or her own diagnosis support algorithm.

The method of providing a clinical diagnosis support service according to the third embodiment of the present disclosure starts with generating examination information in the examination device 10 by allowing the diagnostician to photograph an inside of the patient's oral cavity with the examination device 10 (S501).

Subsequent to generating the examination information by the examination device 10, the examination device 10 transmits the generated examination information to the diagnosis support system 120 (S502).

Then, the diagnosis support system 120 generates diagnosis support data through a reference algorithm uploaded in the server (S503).

Subsequent to step S503, the diagnosis support system 120 provides diagnosis support data generated through the reference algorithm to the diagnostician terminal 20 (S504).

Subsequent to providing, by the diagnosis support system 120, diagnosis support data generated through the reference algorithm to the diagnostician terminal 20 (S504), when the diagnostician does not like the provided diagnosis support data or to generate his or her own diagnosis support algorithm, factors determined in the reference algorithm are added or deleted or the algorithm is changed and set through the diagnostician terminal 20 (S505).

When the diagnostician terminal 20 changes and sets the algorithm (S505), the diagnosis support system 120 generates modified diagnosis support data through the changed diagnosis support algorithm (S506).

Then, the diagnosis support system 120 provides modified diagnosis support data to the diagnostician terminal 20 (S507), thereby completing the method of providing a clinical diagnosis support service according to the third embodiment of the present disclosure.

[Diagnosis Tendency]

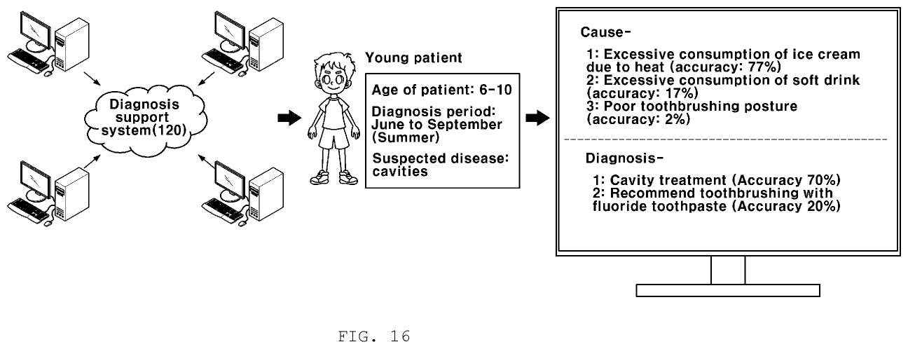

FIG. 16 is a diagram showing a process of providing a diagnosis tendency to a diagnostician by referring to a cumulative database (diagnosis support algorithm or diagnosis support data) previously stored in the diagnosis support system of the present disclosure.

Referring to FIG. 16, the diagnosis support system 120 receives a very large amount of data (diagnosis support algorithm or diagnosis support data) from the diagnostician terminals 20 of the diagnosticians who use the diagnosis support service of the present disclosure, wherein big data is constructed based on these data, and through the big data, tendencies in diagnoses made by a plurality of diagnosticians according to the patient's gender, age, and diagnosis period are obtained and provided to a user. As a specific example, assuming that the diagnostician is diagnosing a pediatric patient, the diagnostician may select and input the pediatric patient's information (age, gender, diagnosis period, suspected disease) and transmit the information to the diagnosis support system 120, which analyzes the trends of patients with similar age, same gender, diagnosis period, and suspected disease as the pediatric patient through big data built in the server, and provides the analyzed result values to the user to support diagnosis. As a more specific example, assuming that the young patient is 7 years old, the diagnosis period is July (summer), and the suspected disease is dental caries (cavities), the diagnostician may receive big data analysis (diagnosis tendencies) showing that younger patients of similar age have been diagnosed with cavities mainly due to excessive consumption of ice cream due to heat.

[Method of Providing Clinical Diagnosis Support Service—\textcircled{4}: Fourth Embodiment]

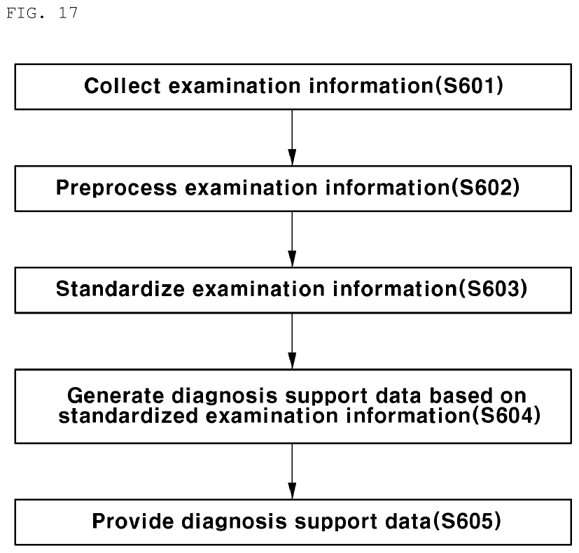

FIG. 17 is a diagram specifically showing a method of providing a clinical diagnosis support service according to a fourth embodiment of the present disclosure.

The method of providing a clinical diagnosis support service according to the fourth embodiment of the present disclosure is an example of a method in which a preset diagnosis support algorithm of the diagnosis support system 120 generates diagnosis support data and provides the generated diagnosis support data.

The method of providing a clinical diagnosis support service according to the fourth embodiment of the present disclosure starts with collecting, by the diagnosis support system 120, examination information from the examination device 10 (S601).

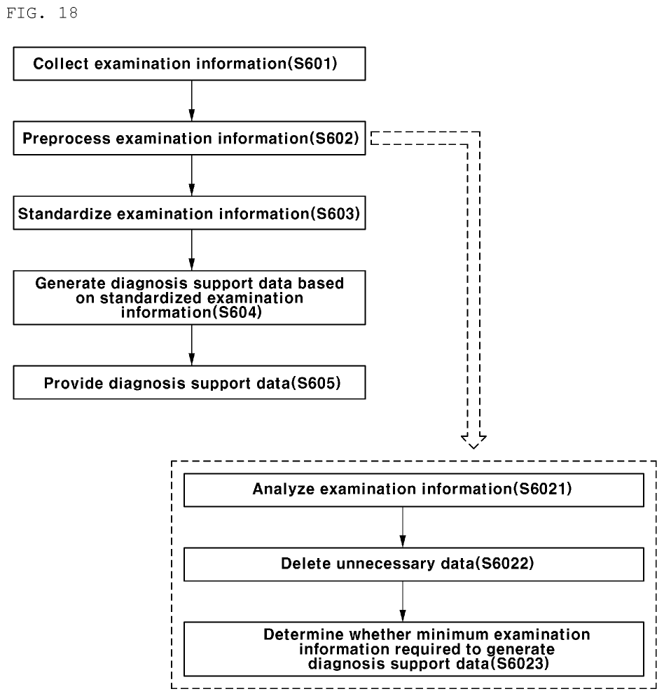

When the diagnosis support system 120 collects examination information from the examination device 10 (S601), the diagnosis support system 120 preprocesses the examination information (S602). Here, referring to FIG. 18, the preprocessing of the examination information (S602) may include analyzing the examination information (S6021), deleting unnecessary data (S6022), or determining whether the minimum examination information required to generate diagnosis support data has been acquired (S6023).

The analyzing of the examination information (S6021), which is collecting or analyzing all information that can be included in the diagnosis support data based on the examination information, may perform analysis by prioritizing the factors (disease factors, risk factors, or protective factors) set in the step S303 (see FIG. 13).

The deleting of unnecessary data (S6022), which is minimizing the data resources on the system to reduce the amount of data stored in the system, and distinguishing between necessary data and unnecessary data to delete unnecessary data so as to improve the speed of data transmission and reception, wherein necessary data and unnecessary data may be classified based on factors set by the user, or data that is analyzed as unnecessary by the diagnosis support system 120 may be classified as unnecessary data.

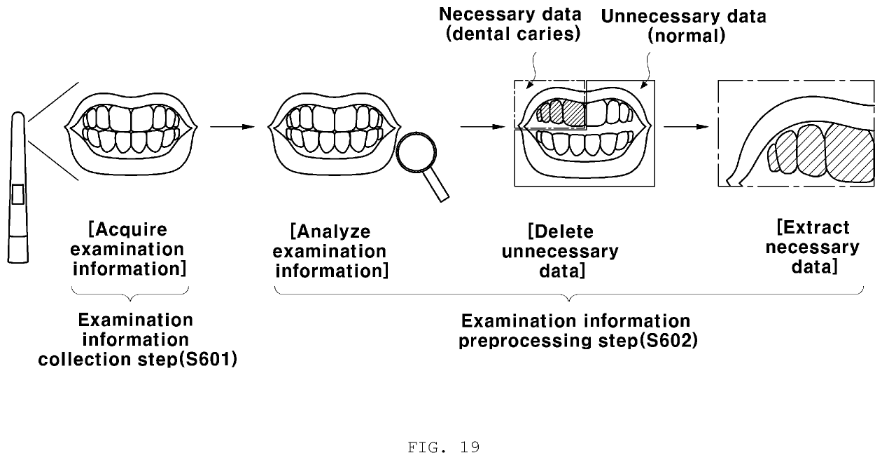

Meanwhile, FIG. 19 is a diagram showing a process of distinguishing between necessary data and unnecessary data and removing the unnecessary data. Referring to FIG. 19, the diagnosis support system 120 may collect the examination information from the examination device 10, and classify a portion of the teeth suspected of having a disease (e.g., cavities) as necessary data, and a portion of normal teeth not suspected of having a disease as unnecessary data from among the analyzed results. Unnecessary data classified in this way will be deleted by the diagnosis support system 120, and the examination information will be preprocessed so that only necessary data remains.

The determining of whether the minimum examination information required to generate diagnosis support data has been acquired (S6023) determines whether the minimum examination information required to generate diagnosis support data has been acquired since the information included in the examination information may be insufficient to generate diagnosis support data, or the diagnostician may have incorrectly used the examination device 10 and incorrect examination information may have been acquired, and guides to re-acquire examination information through the examination device 10 without generating diagnosis support data when the minimum examination information is not acquired.

Referring again to FIG. 17, when the diagnosis support system 120 preprocesses the examination information (S402), the diagnosis support system 120 standardizes the examination information (S403). Here, standardizing the examination information may mean converting the examination information into a dental data code as shown in FIG. 5.

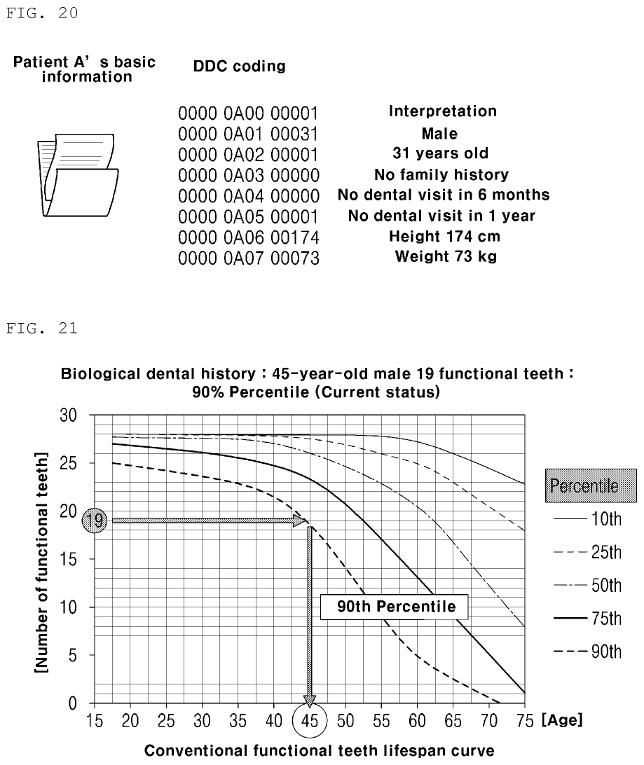

The dental data code in the diagnosis support system 120 may refer to the dental data code generated and processed by the teeth information processing system 110 described above, but in order to more accurately perform clinical diagnosis for patients, in addition to coordinate codes, examination codes, and evaluation codes, information on the patient may be included as shown in FIG. 20. For example, dental data codes may include the patient's gender, age, marital status, dental visit history, body size, or family history.

Referring again to FIG. 17, when the diagnosis support system 120 standardizes the examination information into a dental data code (S603), the diagnosis support system 120 generates diagnosis support data based on the standardized examination information (S604), and provides the generated diagnosis support data to the user terminal 30, thereby completing the method of providing a clinical diagnosis support service according to the fourth embodiment of the present disclosure.

[Method of Providing Clinical Diagnosis Support Service—\textcircled{5}: Fifth Embodiment]

The clinical diagnosis support service according to the fifth embodiment of the present disclosure provides a functional tooth lifespan curve to a diagnostician to guide him or her to perform an efficient clinical diagnosis. Here, a functional tooth refers to a tooth that can perform its original function (e.g., masticatory function), and may be said to be a normal tooth that does not suffer from diseases such as dental caries, periodontal disease, or tooth fracture. In addition, the functional tooth lifespan curve is a curve that shows the number of remaining functional teeth predicted according to the patient's lifespan by determining the patient's age and the current number of functional teeth. This functional tooth lifespan curve does not simply provide a functional tooth lifespan curve of the patient being diagnosed, but also provides an average functional tooth lifespan curve of normal patients, and thus may be used to remind patients of a risk of the teeth condition.

[Functional Tooth Lifespan Curve]

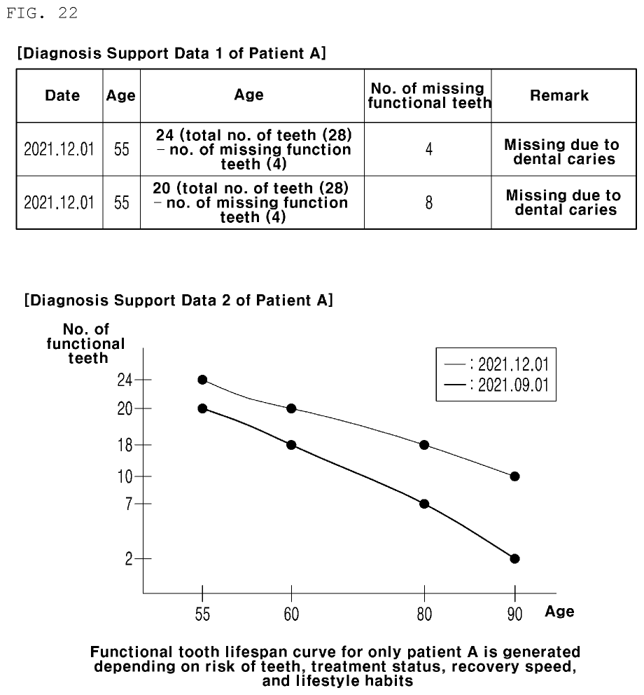

FIG. 21 is a diagram showing a conventional functional teeth lifespan curve. Referring to FIG. 12, a horizontal axis (X-axis) of the functional tooth lifespan curve represents the patient's age, and a vertical axis (Y-axis) represents the number of functional teeth of the patient. A conventional functional tooth lifespan curve may consist of a plurality of percentile curves. These percentile curves are curves that predict biological tooth age, such as the top 10%, 25%, 50% (average percentile), 75%, and 90% percentile, using the number of functional teeth according to the patient's age.

As an example of a process in which a patient's clinical diagnosis is carried out through this functional tooth lifespan curve, the diagnostician may first determine the patient's age and number of functional teeth to find a percentile curve corresponding to a functional tooth lifespan curve table (referring to FIG. 21, if a 45-year-old man currently has 19 functional teeth, then this man corresponds to the 90% percentile curve), and in case of finding a percentile curve of the patient to be diagnosed, he or she may perform a diagnosis on the patient by informing the patient of the number of functional teeth in the future that can be predicted through the percentile curve, or comparing with the 50% percentile corresponding to the normal category to explain how the relevant patient differs from normal patients.

However, on the other hand, it is difficult to predict the exact number of functional teeth in the conventional functional tooth lifespan curve as shown in FIG. 21 because it simply determines whether a tooth is a functional tooth or not based on whether or not a tooth requires extraction. In other words, in order to measure the number of functional teeth, the conventional functional tooth life curve simply considers whether the tooth in question is a tooth that needs to be extracted, but does not consider teeth expected to be extracted due to disease (dental caries, periodontal disease, tooth fracture, etc.), making it difficult to accurately predict remaining functional teeth.

On the contrary, the diagnosis support system 120 of the present disclosure may additionally analyze the ‘number of missing functional teeth’ in order to provide a more accurate functional tooth lifespan curve to the diagnostician. Here, a missing functional tooth refers to a tooth that is currently not extracted or a tooth that is expected to be unable to perform its natural function, such as chewing, due to a disease or illness, which includes teeth that have the potential to become functional teeth again. This number of missing functional teeth is subtracted from the total number of teeth (28) to calculate an accurate number of functional teeth. In other words, the diagnosis support system 120 according to the present disclosure may further calculate a patient's ‘number of missing functional teeth’ by referring to the diagnosis support data described above, and further reflect the number of missing functional teeth from a conventional functional tooth lifespan curve obtained from a result of observing the patient, thereby conveying more accurate dental health status to the patient.

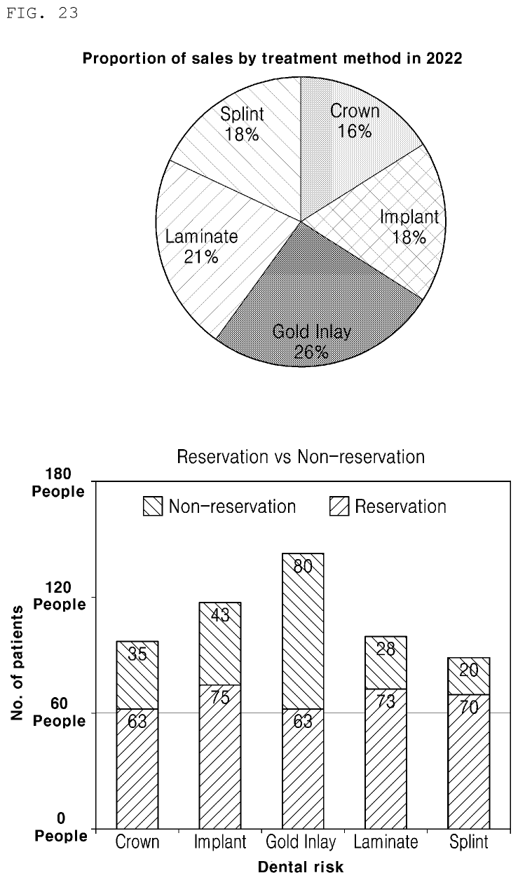

FIG. 22 is a diagram showing data related to functional teeth included in the diagnosis support data generated by the diagnosis support system 120 of the present disclosure. Referring to FIG. 22, ‘patient A's diagnosis support data 1’ shows diagnosis support data related to functional teeth through a table. The number of missing functional teeth provided in the diagnosis support data is a result value of classifying teeth that are dangerous enough to require extraction among the teeth analyzed by the diagnosis support system 120 through the diagnosis support algorithm. When the diagnosis support system 120 calculates the number of missing functional teeth in this way, a more accurate number of functional teeth may be obtained by subtracting the number of missing functional teeth from the total number of teeth (28). For reference, the diagnostician may arbitrarily set a risk of teeth that can be classified as missing functional teeth through the diagnosis support system 120, thereby generating a functional tooth lifespan curve that reflects the diagnostician's individual experience, knowledge, and intention.

Meanwhile, ‘patient A's diagnosis support data 2’ shown in FIG. 22 is a functional tooth lifespan curve of patient A formed based on a result value of the diagnosis support system 120 that analyzes the patient's examination information. The lifespan curve may be updated each time the patient revisits the hospital and is diagnosed, and the functional tooth lifespan curve for only patient A is formed depending on a risk of the patient's teeth, treatment status, recovery speed, and lifestyle habits. In addition, the functional tooth lifespan curve of the present disclosure, similar to the conventional functional tooth lifespan curve, may be provided with a normal category functional tooth lifespan curve, an upper percentile curve, and lower percentile curves.

A process of performing a diagnosis for a patient using ‘patient A's diagnosis support data 1’ and ‘patient A's diagnosis support data 2’ shown in FIG. 22 may be carried out as follows. When patient A visits the hospital on ‘Sep. 1, 2021’, the diagnosis support system 120 may provide the diagnostician through diagnosis support data that patient A has 8 missing functional teeth, and the diagnostician may convey the risks to the patient and perform dental procedures based on the patient's consent and the diagnostician's judgment. Meanwhile, when patient A revisits the hospital on ‘Dec. 1, 2021’, 3 months later, the diagnostician may acquire updated diagnosis support data through the diagnosis support system 120, and through this, the diagnostician may not only confirm that the number of patient A's missing teeth has been reduced to four in three months, but also confirm that a degree of decline in patient A's functional tooth curve has improved. Accordingly, the diagnostician may perform a diagnosis for patient A, saying, “If you maintain this lifestyle and continue treatment, it will help improve your dental health.” In this way, the functional tooth lifespan curve provided to the diagnostician through the diagnosis support system 120, especially the functional tooth lifespan curve information in which the patient's examination results are reflected in the diagnosis support data and the number of missing functional teeth not only conveys the patient's current oral cavity health status accurately to both the diagnostician and the patient, but also has an effect of enabling them to gauge whether the current treatment or management processes are progressing appropriately.

[Functional Tooth Lifespan Curve According to Systemic Disease]

Meanwhile, the diagnosis support system 120 of the present disclosure may generate a new functional tooth lifespan curve based on systemic diseases that may cause dental disease in a patient. In other words, the diagnosis support system 120 may classify patients with at least one systemic disease such as obesity, diabetes, heart disease, and arteriosclerosis that can cause dental disease according to the type of disease to form each population (obesity population, diabetes population, etc.), and calculate an average value of the functional tooth lifespan curves of the formed each population so as to generate a new functional tooth lifespan curve based on systemic disease. The generated new functional tooth lifespan curve may be used as an excellent reference when making functional tooth-related diagnoses for patients with systemic diseases. The reason why it is possible to generate functional tooth life curve information according to systemic disease is because the diagnosis support system 120 according to the present disclosure may ultimately construct big data by accumulating information from many patients about their individual diseases and the examination information generated when the patient is examined by a medical examiner.

[Method of Providing Clinical Diagnosis Support Service—\textcircled{1}: Sixth Embodiment]

The diagnosis support system 120 according to a sixth embodiment of the present disclosure may provide not only services related to the diagnosis of patients, but also services that provide information related to sales of the hospital operated by the diagnostician.

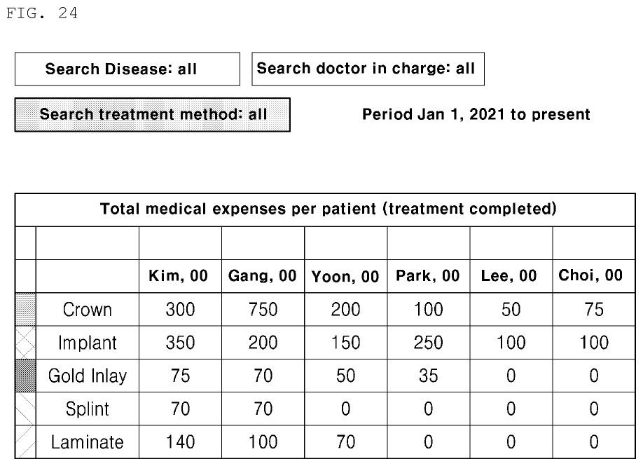

Referring to FIG. 23, the diagnosis support system 120 of the present disclosure may provide the diagnostician with a proportion of sales by treatment method for the relevant year and month and the number of patients according to treatment method. For reference, when considering the number of patients according to treatment method, patients who have made a reservation for treatment (or examination) are distinguished from those who have not made a reservation, thereby allowing the diagnostician to more conveniently check future profit forecasts and current sales status (or reservation status).

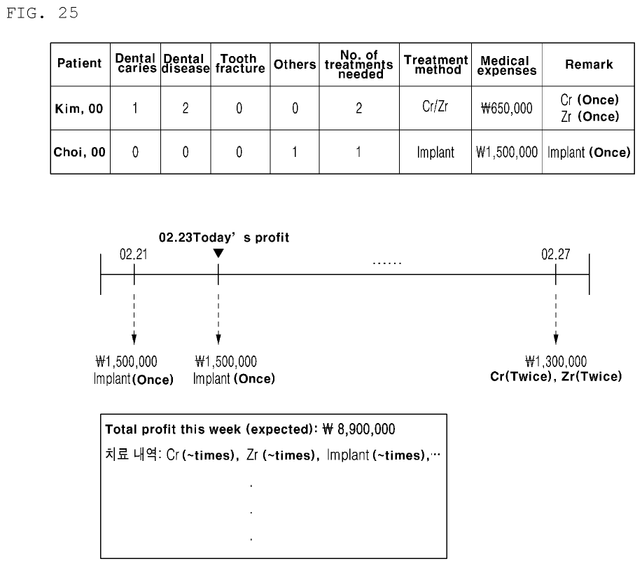

Referring to FIG. 24, the diagnosis support system 120 of the present disclosure may classify the treatment method performed for each patient and the corresponding medical expenses for each patient to provide them to the diagnostician. In this manner, the diagnosis support system 120 of the present disclosure may record past treatments performed by the diagnostician in the form of data to be used as a sales ledger.

In addition, the diagnosis support system 120 of the present disclosure may organize the recorded data in various ways (number of patients according to treatment method, classification of medical expenses for each individual patient) to help operate the hospital efficiently according to the organized resultant outputs. For example, if dental caries treatment provided at a hospital accounts for more than 70% of entire treatment methods performed, then the hospital may purchase more equipment related to dental caries treatment methods or conduct marketing related to the dental caries treatment methods to make the hospital more efficient to help operate the hospital efficiently.

Meanwhile, the diagnosis support system 120 of the present disclosure may predict information on future profits and provide the predict information to the diagnostician. Specifically, the diagnosis support system 120 may analyze a patient's examination information to provide the diagnostician with information on what dental treatment method the patient needs and how many more treatments are needed in the future, wherein when the diagnostician sets up a price corresponding to each treatment method, the diagnosis support system 120 may not only provide information on medical expenses corresponding to the treatment method required by the patient in the diagnosis support data, but also provide information on expected total future medical expenses that can be achieved by treating current patients in the future to the diagnostician.

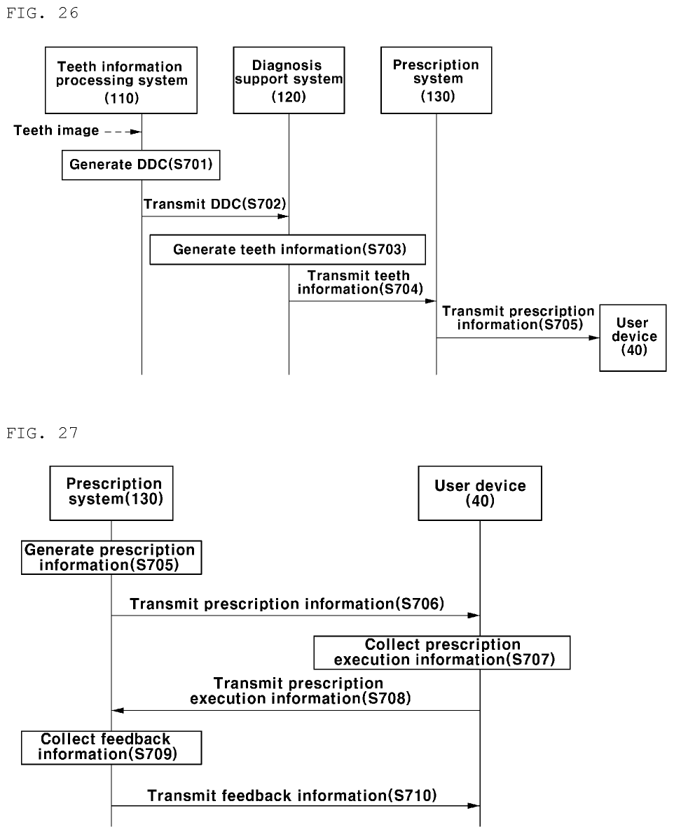

FIG. 25 is a diagram showing a form in which diagnosis support data includes data related to medical expenses. Referring to FIG. 25, the diagnosis support data includes a patient's name, a teeth condition, a number of necessary treatments, a treatment method, and a medical expense. Additionally, the diagnosis support data may include information on total revenue predicted on either a weekly or monthly basis. Specifically, the diagnosis support data may calculate not only past/current profits but also future profits within a reference period (week/month), thereby providing a total expected profit within that period to a diagnostician. For reference, the future profits referred to herein will be profits calculated by considering treatments previously reserved by a patient or an expected number of treatments required for one patient.

In the above, the method of providing a clinical diagnosis support service, which is a second step of the medical service of the present disclosure (\textcircled{2}) has been described.

Next, the prescription service provision method, which is a third step of the medical service of the present disclosure (\textcircled{3}) will be explained.

<Method of Providing Prescription Service (\textcircled{3})>

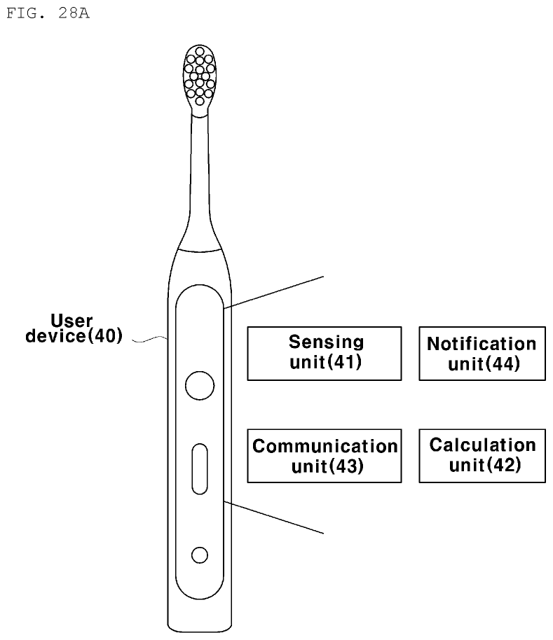

FIGS. 26 to 32 are diagrams for explaining the prescription service provision method (\textcircled{3}).

[Prescription System 130]

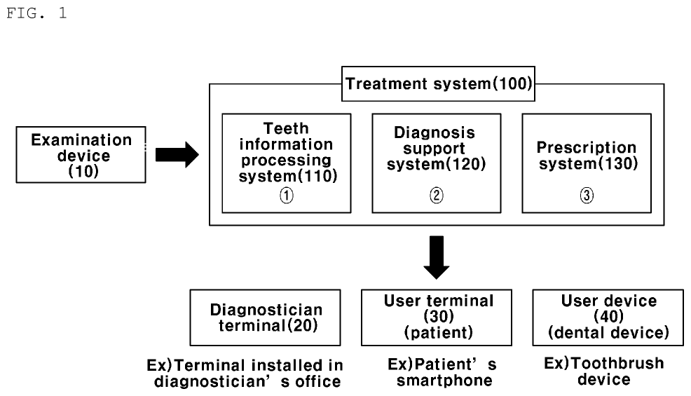

Referring to FIG. 1, the prescription system 130 of the present disclosure may receive teeth information from the examination device 10 or another system (teeth information processing system 110, diagnosis support system 120). For reference, it is to be understood that the teeth information mentioned here may refer to the examination information previously described in the diagnosis support system 120. Next, prescription information may be generated by performing calculation according to a series of algorithms based on the received teeth information, and the generated prescription information may be provided to various types of diagnostician terminals 20, user terminals 30, and user devices 40. For reference, the diagnostician terminal 20 and the user terminal 30 may be a smartphone carried by the user or a computing device used by the diagnostician during treatment, and the user device 40 may be a toothbrush device or a treatment device.

[Relationship Between Prescription System 130 and Other Systems]

The prescription system 130 according to the present disclosure may receive teeth information (or examination information) from another system (teeth information processing system 110, diagnosis support system 120), wherein the teeth information may include a dental data code, or may include a result value calculated by the diagnosis support system 120. The dental data code (DDC) or the result value from the diagnosis support system 120 will be further described later, but the dental data code or result values from the diagnosis support system 120 may all include at least one of information that can specify the location of an individual tooth (tooth location information) and information that distinguishes the condition of the tooth (tooth condition information), and logically and naturally, the tooth information may also include this information. The tooth location information or tooth condition information may correspond to information essential for the prescription system 130 according to the present disclosure to generate prescription information. In addition, in FIG. 1, it will be understood that the prescription system 130 according to the present disclosure receives a dental data code or calculation result value from separate systems, for example, the teeth information processing system 110 and/or the diagnosis support system 120, but the present disclosure is not necessarily limited thereto, and it is to be understood that the prescription system 130 may directly generate dental data codes or function as a diagnosis support system to generate a result value. In this case, it should be understood that the prescription system 130 does not receive teeth information from the outside, but generates and acquires teeth information on its own.

[User Device 40]

Meanwhile, for the user device 40, a toothbrush device, an oral cleaning device, and the like, used by an individual user who receives prescription information from the prescription system 130 for dental care, may also be included in the scope of the user device 40 mentioned in this detailed description when provided with a network function and an electronic computing function.

For reference, in order to easily understand the prescription service provision method that will be described later, it should be understood that the user device 40 is distinguished from the diagnostician terminal 20 and the user terminal 30. Specifically, considering that the diagnostician terminal 20 refers to a computing device installed in a medical office, which is a device that outputs information necessary for medical treatment, and the user terminal 30 is a commonly used smart phone, the user device 40, the diagnostician terminal 20, and the user terminal 30 may be easily understood in a distinguished manner.

[Method of Providing Prescription Service Using Teeth Information \textcircled{1}—Seventh Embodiment]

FIG. 26 is a diagram specifically showing a seventh embodiment in a method of providing a prescription service using teeth information according to the present disclosure. Referring to FIG. 26, steps actually performed by the prescription system 130 according to the present disclosure start from step S704 in the drawing, but for an overall understanding of the disclosure, they will be described from an initial process of generating a dental data code.

The method of providing a prescription service using teeth information may first start with a step (S701) of generating a dental data code (DDC). This step may be carried out by the teeth information processing system 110, and the teeth information processing system 110 may first receive and store a subject person's teeth image prior to generating the dental data code (DDC). In this case, the teeth image may be acquired by an intraoral-only camera (scanner) or a device that can generate an image by photographing a certain object, even if it is not necessarily intended for oral cavity only.

Subsequent to step S701, the previously generated dental data code (DDC) may be transmitted to the diagnosis support system 120 (S702), and the diagnosis support system 120 may generate teeth information (herein, it is understood that the teeth information may be diagnosis support data) using the received dental data code (S703). The teeth information generated by the diagnosis support system 120 may include at least one of location information capable of identifying the location of an individual tooth and condition information capable of identifying the condition of the tooth, and the information may essentially be the same as the information included in the data code (DDC). Meanwhile, the teeth information may further include information processed and added by the diagnosis support system 120, and for example, may further include medical information on respective teeth that are learned and analyzed by an artificial intelligence algorithm of the diagnosis support system 120 (herein, it is understood that the artificial intelligence algorithm may be the diagnosis support algorithm described above).

Meanwhile, subsequent to step S703, teeth information may be transmitted to the prescription system 130 (S704), and the prescription system 130 may generate prescription information based thereon (S705).

The prescription information is information that can be displayed through the diagnostician terminal 20 or the user terminal 30 or utilized to induce a specific operation of the user device 40, which may be transmitted from the prescription system 130 according to the present disclosure to respective devices or terminals through a network (S706).

The prescription information, like the teeth information, may include at least one of location information and condition information of teeth, but the location information or condition information included in the prescription information may be different in format from those included in the teeth information. This is because the prescription information and the teeth information may be data in different formats, wherein it is understood that those formats may be freely determined depending on the designer's choice.

On the other hand, the prescription information may have the form of numerals, letters, a combination of numerals and letters, images, videos, or codes that are readable by a certain device.

For example, if the prescription information is intended for the user to view through a display screen, such as a smartphone, then the prescription information may be generated in the form of numerals, letters, a combination of numerals and letters, images, and videos. The prescription information transmitted to the user's smartphone may be displayed on a display screen through a dedicated application (APP) or by a unique function of the smartphone, and the user may confirm a prescription that suits his or her teeth and then perform a prescription action (teeth management action). For a more specific example, the prescription information may include a prescription for brushing teeth, including information on which teeth and how long the user should brush, information on a brushing direction, and between which teeth the user should floss, and this prescription information may be displayed in the form of an image or video so as to allow the user to easily recognize.

For another example, the prescription information may include information that can be referenced for driving a certain dental care device, for a more specific example, a toothbrush device. The user may brush his or her teeth using a toothbrush device capable of detecting posture, and in this case, the toothbrush device may receive prescription information from the prescription system 130 and then be driven by referring to information included in the prescription information. In this case, various information may be stored in the prescription information in the form of codes that can be read by the toothbrush device, and may herein include information that can be referred to when the toothbrush device is driven, such as location information of teeth, posture information of teeth, and specific drive (vibration, sound, etc.) information mapped when the toothbrush is in a specific posture.

On the other hand, the prescription information may further include information related to a dental prescription, such as a prescription for pharmaceutical treatment, a prescription including whether surgery is necessary, or an instruction for dental care. On another hand, artificial intelligence algorithms may also be utilized in the process of generating prescription information by the prescription system 130. In other words, the prescription system 130 may learn past prescription information or receive new prescription information and analyze it, and when teeth information in a similar form as the prescription information generated in the past is input, may be implemented to generate similar prescription information based on what has been learned.

[Method of Providing Prescription Service Using Teeth Information \textcircled{1}—Eighth Embodiment]

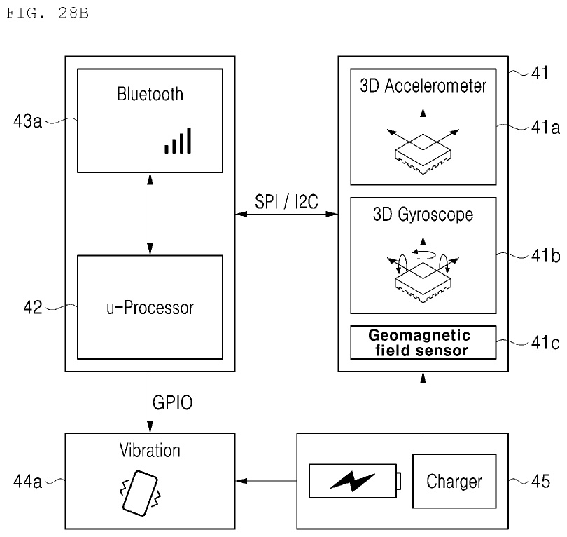

Meanwhile, FIG. 27 shows an eighth embodiment, which is an additional embodiment continuing from FIG. 26, describing a process in which after prescription information is transmitted to the user device 40 by the prescription system 130, when prescription execution information is collected and received by the user device 40, the prescription system 130 generates and provides feedback information based thereon.

Referring to FIG. 27, the prescription system 130 receives prescription execution information collected by the user device 40, wherein it is to be understood that the prescription execution information includes information on how the user actually has performed dental care or an action for dental treatment according to the previously provided prescription information.

For example, the prescription execution information collection step (S707) of FIG. 27 may be a step of receiving an input directly by a user about how he or she took care of teeth according to the previous prescription information directly through a device, that is, a smartphone. For a more specific example, when a preset period (1 hour, 1 day, 1 week, 1 month, etc.) has passed after prescription information is received from the prescription system 130, a dedicated application installed on a smart phone may display a survey on how much the user has followed the prescription information and has taken care of his or her teeth, and transmit, after receiving the user's input, the collected prescription execution information to the prescription system 130. In another manner, the prescription execution information collection step (S707) may be also performed by a device provided with a sensor capable of collecting posture, a sensor capable of collecting information on movement, and a sensor for calibration, such as a toothbrush device, and may also be performed by collecting the posture and movement information of the device in real time in case where, when the user brushes teeth with a toothbrush device, the information that can be collected from the brushing action is detected by the sensors.

For reference, the prescription execution information mentioned above illustrates simple examples to aid the understanding of the disclosure, wherein it is to be understood that the prescription execution information may include information corresponding to actions taken by the user to manage or treat his or her teeth, in particular, information that can be input, sensed, and received through the user device 40.

Meanwhile, subsequent to step S707, the prescription system 130 may receive prescription execution information from the user device 40 (S708) and then generate feedback information corresponding thereto (S709). Feedback information may be information indicating how well the user has managed his or her teeth according to the prescription when compared to the prescription information provided to the user device 40. For example, the prescription system 130 may compare a survey result directly entered by the user with the previously provided prescription information to calculate which prescription items have been fulfilled and how much they have been fulfilled. In addition, the prescription system 130 may compare posture information and movement information detected by the user device 40 with the prescription information provided in advance, thereby calculating a degree of fulfillment of the prescription, such as whether the corresponding user device 40 has been properly driven according to the prescription, in other words, whether the user has moved the toothbrush device according to the prescription information.

Next, the feedback information generated in this way may be provided back to the user device 40 to lead the user to understand how he or she is taking care of his or her teeth.

With reference to FIGS. 26 and 27 as described above, an entire prescription service provision process involving the prescription system 130 according to the present disclosure has been described.

[Configurations of Various User Devices 40]

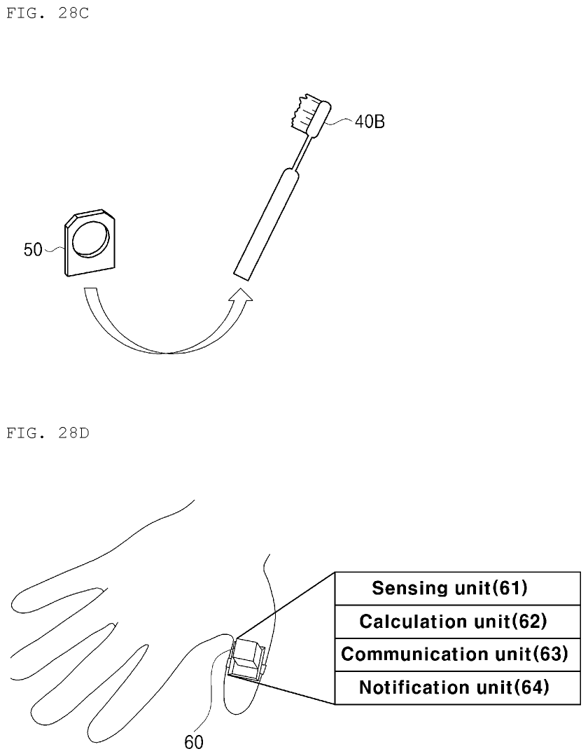

FIG. 28A is a diagram for explaining the user device 40 that receives prescription information from the foregoing prescription system 130, particularly a toothbrush device, as an example.

Referring to FIG. 28A, the user device 40 may include a sensing unit 41, a calculation unit 42, a communication unit 43, and a notification unit 44. However, in this case, the configuration of the user device 40 is only a main configuration for achieving an object of the present disclosure, and it is to be understood that some configurations may be added or deleted as needed, and the role performed by one configuration may also be performed by another configuration.

The sensing unit 41 may be a sensor that detects a prescribed movement or posture. The sensing unit 41 may include, but is not limited to, a gyro sensor for measuring angular velocity, an acceleration sensor for measuring acceleration, a geomagnetic field sensor for calibrating the sensors, and a pressure detection sensor for detecting pressure, and there is no limit to the type as long as it can detect movement and posture.

The user device 40 may detect the user's movement or action through the sensing unit 41, and for example, the user device 40 may detect at which angle and posture the user brushes his or her teeth through the gyro sensor of the sensing unit 41, detect the intensity of toothbrushing through an acceleration sensor, or detect the pressure of whether the toothbrush has properly contacted the teeth through a geomagnetic field sensor and a pressure sensor for calibrating the sensors.

The calculation unit 42, which is an element that processes a program, including a central processing unit, may generate prescription execution information based on data sensed by the sensing unit 41. In this case, the meaning of generating prescription execution information may include packaging the collected information and converting the collected information into a format suitable for transmission to the prescription system 130.

Meanwhile, the prescription execution information is information on the user's movement or action generated when the user uses the user device 40. For example, this may be information on whether the user is brushing his or her teeth, information on an angle or posture at which the user performs tooth brushing, and information on the intensity of tooth brushing. Additionally, the action information may be information used to determine whether a target value corresponding to feedback information, which will be described later, has been reached.

The communication unit 43 is an element that allows the user device 40 to have a network function, which performs the role of receiving prescription information from the prescription system 130, and transmitting prescription execution information to the prescription system 130. The communication unit 43 may not necessarily be directly connected to the prescription system 130 over a network, and an additional intermediate component such as a smartphone and a local computer may also be present therebetween. For example, the communication unit 43 may perform Bluetooth communication to enable near field communication with a smartphone, and may transmit data to the prescription system 130 and over the network via the smartphone.

The notification unit 44 is configured to output at least one of light, sound, or vibration. Specifically, when there is prescription information generated by the prescription system 130 or feedback information to be described later, the notification unit 44 may execute a specific notification function of the user device 40 based on this information, for example, may inform whether the user has properly followed a prescription instruction, such as whether the toothbrush device is moving according to the prescription information and whether the intensity of toothbrushing is appropriate.

For reference, FIG. 28B shows a more specific implementation example of the user device 40 that receives prescription information from the prescription system 130. Referring to FIG. 28B, the user device 40 may include a sensing unit 41, more specifically, a 3D acceleration sensor 41a, a 3D gyro sensor 41b, and a geomagnetic field sensor 41c. Additionally, the user device 40 may include a calculation unit 42 and a Bluetooth communication unit 43a, and the information acquired by the sensing unit 41 may be processed through the calculation unit 42 and the Bluetooth communication unit 43a or transmitted or received between other external devices. In addition, the user device 40 may further include a notification unit 44, specifically a vibration notification unit 44a, thereby transmitting a notification signal through vibration to the user. Finally, the user device 40 may further include a power supply unit 45 as a means for supplying power, and the power supply unit 45 may be configured to consume stored electrical energy by charging, or connected to an external power source in a wired or wireless manner to supply power to the various components mentioned above.

Meanwhile, an apparatus that receives prescription information from the prescription system 130 does not necessarily need to be a device in the shape of a toothbrush as shown in FIG. 28A or 28B. That is, the shape of the user device 40 is manufactured in the form of a ring or band as shown in FIG. 28C or FIG. 28D, and is similar to the existing toothbrush or scaling handpiece, and may be in the shape of an existing toothbrush, a scaling handpiece, or a device that can be used by attaching it to the user's finger.

FIG. 28C shows a ring-type device 50 that can be used by inserting it into a handle of an existing toothbrush product 40B or the user's finger, in which the ring-type device 50 may include various components, including a sensing unit, within a housing manufactured in a ring shape. If necessary, the ring-type device 50 may be used as a handle portion of the existing toothbrush product 40B, or another dental care or treatment tool, or any other portion to which the ring-type device 50 can be fitted, and may detect movement according to the user's use of the tool to transmit the detected information or data to a nearby user terminal (smartphone), or the like, and this information or data may also be shared with the prescription system 130. For reference, the ring-type device 50 may be implemented in a streamlined form, and one end of the line may be implemented as a USB connectable to a smartphone or local computer.

FIG. 28D shows a band-type device 60 that can be wrapped around the user's finger, wherein the user may wrap the band-type device 60 as if taping the finger so as to allow it to detect the movement of his or her hand. This band-type device 60 may include an elastic band portion, and may further include a separate housing attached to the band portion. As shown in the drawing, this housing may include a sensing unit 61, a calculation unit 62, a communication unit 63, a notification unit 64, and the like, and for a description thereof, the foregoing description in FIG. 28A will be referenced. Meanwhile, in some cases, the band type-device 60 may be provided with the sensing unit, the calculation unit, the communication unit, or the notification unit directly on the band portion without having a separate housing.

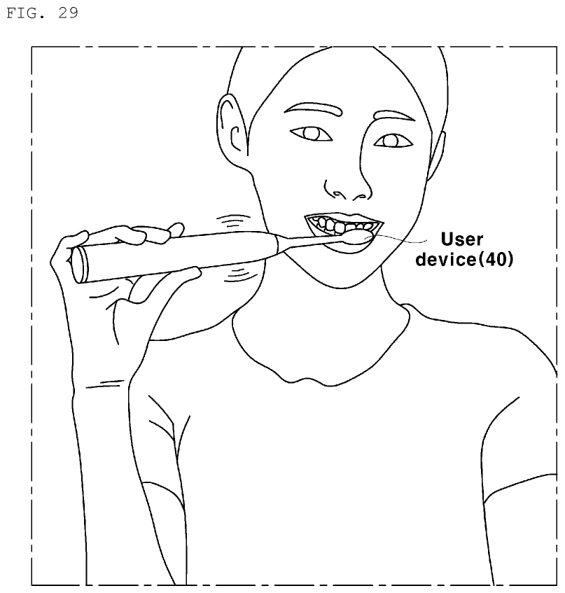

Below, starting from FIG. 29, how a prescription service according to the present disclosure can be specifically applied to real life will be considered.

[Example of Prescription Service Use \textcircled{1}—Ninth Embodiment]

First, FIG. 29 is for explaining a ninth embodiment in which a user brushes teeth using the toothbrush device 40, and specifically shows a shape in which the toothbrush device 40 receives prescription information or feedback information from the prescription system 130 to operate based thereon, and more precisely, a shape in which a vibration or warning sound is output from the notification unit 44 of the toothbrush device 40.

Assuming that the prescription information generated by the prescription system 130 is transmitted to the toothbrush device 40, the prescription information may include location information on a portion of the teeth that must be brushed when the user brushes his or her teeth, or information on the minimum tooth brushing intensity required when the user brushes his or her teeth.

When the toothbrush device 40 receives such prescription information, the prescription system 130 may receive prescription execution information from the toothbrush device 40 to check whether the user has properly brushed his or her teeth according to the prescription information.

Prescription execution information, which is information on actions or movements generated when a user uses the toothbrush device 40, is information that is generated by the sensing unit 41 in the toothbrush device 40, which detects the user's actions or movements, and is generated by the calculation unit 42 based thereon. The prescription execution information may include information on the user's toothbrushing location and posture obtained through the gyro sensor, the acceleration sensor, and the geomagnetic field sensor for calibrating the sensors included in the sensing unit 41, and also include information on the intensity of toothbrushing obtained through a pressure detection sensor.

When the prescription system 130 receives the prescription information generated from the toothbrush device 40, the prescription system 130 generates feedback information based on the received prescription execution information and the previously provided prescription information.

The feedback information herein may be evaluation information on toothbrushing by comparing the prescription information generated first by the prescription system 130 with the prescription execution information collected later. For example, assuming that the prescription system 130 provides the user with prescription information such as ‘brush your teeth at least 20 times’ and that the user brushes his or her teeth only 10 times, the toothbrush device 40 may transmit prescription execution information on the user having brushes the teeth only 10 times to the prescription system 130, and the prescription system 130 may compare and calculate the prescription execution information with the original prescription information to generate and provide feedback information indicating that more than 10 tooth brushings are needed to the user.

As shown in FIG. 29, the feedback information may be provided to the user through the notification unit 44 of the toothbrush device 40. For example, vibration may be generated through the notification unit 44 until the feedback information of ‘brushing teeth more than 20 times’ provided by the prescription system 130 is satisfied, or when the user does not respond to the feedback information and stops brushing the teeth, a message such as “more than 10 tooth brushings are further needed” may be output through the notification unit 44. The notification unit 44 may include any means for outputting at least one light, sound, or vibration, and may specifically include display means such as OLED or LCD. Meanwhile, for another example, when a specific tooth, such as a tooth with cavities or a tooth with a high risk of plaque, needs more tooth brushing, the vibration or warning sound of the notification unit 44 may assist in locating the specific tooth.

[Example of Prescription Service Use \textcircled{2}—Tenth Embodiment]

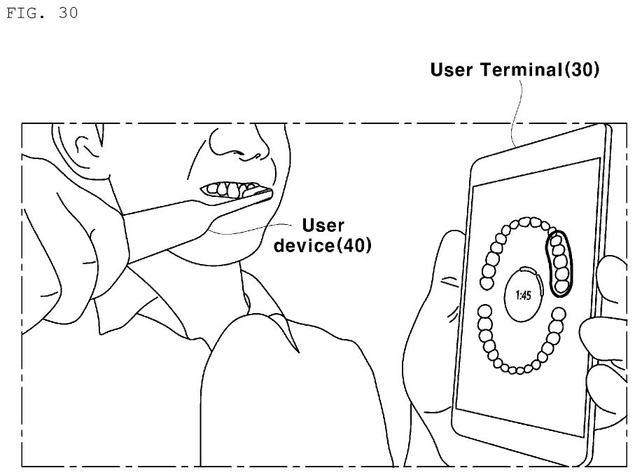

Next, FIG. 30 is a diagram for explaining a tenth embodiment in which a user brushes his or her teeth while looking at the user terminal 30 (e.g., a smartphone), wherein specifically, prescription information or feedback information generated from the prescription system 130 is output through the application of the user terminal 30 to allow the user to check a toothbrushing process while looking at the user terminal 30 in real time.

The tenth embodiment of the present disclosure is similar to the ninth embodiment in that it generates teeth information and generates prescription information and feedback information based thereon, but differs in that it displays the information for the user using an application.

The application may be an application that can read and receive data about the DDC generated from the teeth information processing system 110 and the teeth information generated from the diagnosis support system 120, which is an application that delivers information tailored to the user's teeth, such as information on the condition of the user's teeth, information on dental care instructions, and information on dental prescriptions.

According to FIG. 30, the user may check a toothbrushing progress process in real time using the application. Specifically, information displayed on the application may be a 3D teeth image, and the 3D teeth image is a teeth image corresponding to the user's teeth information, which is an image showing a location of the disease of the user's teeth, a risk of the disease, and the like. While looking at the 3D teeth image, the user may identify teeth that need more toothbrushing and may receive information on a number, a time period, and the like needed for toothbrushing.

Additionally, the application may be used in various ways for the user's dental health.

[Example of Prescription Service Use \textcircled{3}—Eleventh Embodiment]

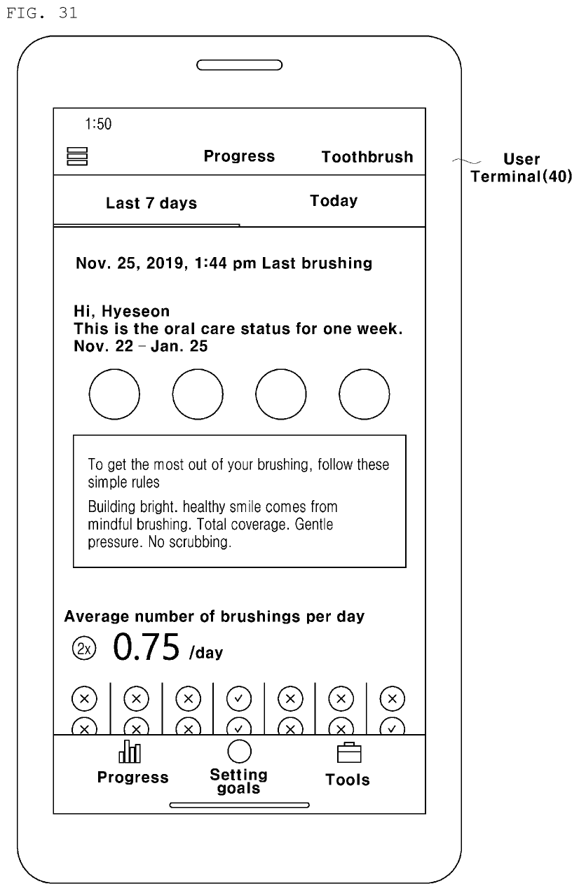

Meanwhile, FIG. 31 shows an eleventh embodiment in which a user using the toothbrush device 40 receives information on a dental care status from the prescription system 130 through a smartphone application. According to FIG. 31, the application may display the user's dental care status based on prescription information or feedback information generated by the prescription system 130 and action information generated by the toothbrush device 40.

For example, the information provided by the application may include text information such as information on the user's shortcomings in past tooth brushing, information on the extent to which the goal presented by feedback information has been reached, and the like, numerical information such as a number of brushings per day, an average number of brushings per week, an average brushing time period per day, an average brushing time period per week, and the like, and image information such as graphs and charts that can be displayed based on the above numerical information.

Moreover, the information provided by the application, which is related to the toothbrush device 40, may include a charging state of the toothbrush device 40, a connection state of the toothbrush device 40, a management state of the toothbrush device 40, whether the toothbrush device 40 is broken, and the like.

[Example of Prescription Service Use—Twelfth Embodiment]

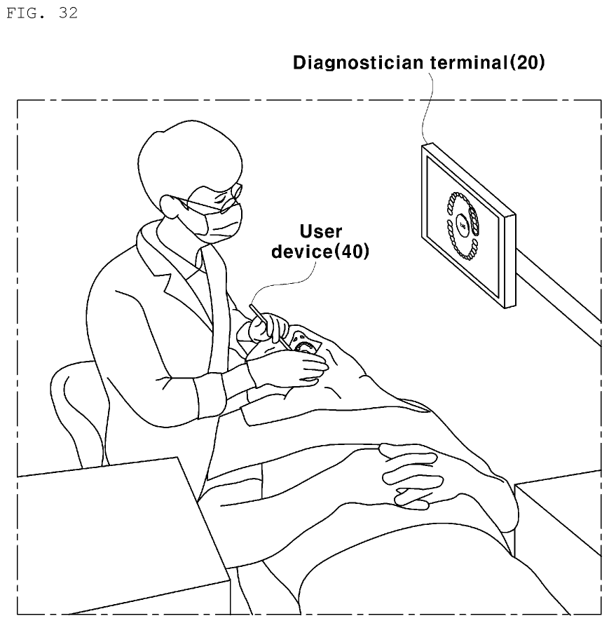

FIG. 32 is a diagram for explaining a twelfth embodiment in which a professional (diagnostician) is performing a procedure on a patient while looking at the diagnostician terminal 20, especially when the user is a professional such as a doctor. Specifically, prescription information or feedback information generated from the prescription system 130 may be displayed on the diagnostician terminal 20, and the user (diagnostician) may perform a procedure by referring to the prescription information or feedback information displayed on the diagnostician terminal 20.

An example of the use in the method of providing the prescription system 130 using teeth information according to the twelfth embodiment of the present disclosure is the same as the eleventh embodiment in generating teeth information, generating prescription information and feedback information based thereon, and displaying the generated information on the diagnostician terminal 40 for reference, but the twelfth embodiment differs in utilizing the terminal 20 and using it for a treatment and procedure.

Information displayed on the diagnostician terminal 20 may be prescription information generated from the prescription system 130. For example, a condition of the patient's teeth, information on a location of disease-infected teeth, information on teeth with a high possibility of disease spread, a difficulty of a procedure for the disease-infected teeth, a risk of the disease-infected teeth, and information on surgical tools required for the procedure may be included therein.

Additionally, information that can displayed on the diagnostician terminal 20 may be feedback information generated from the prescription system 130. For example, the feedback information may be information on a degree of progress when a user (diagnostician) treats or performs a procedure on a patient, or may include additional feedback items or the like according to a specific procedure.

Here, assuming that the user (diagnostician) performs a procedure to remove the patient's tooth, when the prescription system 130 analyzes information on whether the user (diagnostician) has reached a target amount of tooth removal, additional feedback items according to a specific procedure refer to items such as information on the remaining amount of removal to be presented based on the target amount of tooth removal or information on a portion that needs to be removed in case the target amount of tooth removal has not been reached.

[Example of Prescription Service Use \textcircled{5}—Thirteenth Embodiment]

The user device 40 in the method of providing a prescription system using teeth information according to a thirteenth embodiment of the present disclosure includes the sensing unit 41, the calculation unit 42, the communication unit 43, and the notification unit 44, which are the elements of the user device 40 according to the ninth embodiment of the present disclosure, and the roles of the above elements are the same, and thus a description of the same items will be omitted to prevent duplicate description, and only the differences will be described below. For reference, in the case of the user device 40 herein, it is to be understood that a user using the device is a diagnostician, and the previously described examination device 10 is used as the user device 40. In other words, the user device 40 in the method of providing a prescription system using teeth information according to the thirteenth embodiment of the present disclosure is the device 40 used for dental treatment and examination.

The user device 40 according to the thirteenth embodiment receives prescription information or feedback information provided by the prescription system 130 to aid the user (diagnostician) in performing dental treatment and examination more precisely and accurately.

For example, assuming that the user device 40 is a handpiece, the handpiece may also be used to remove teeth so as to form abutments. The abutment is a tooth that serves to support a fixed prosthesis or a removable partial denture (partial denture), and in order to form an ideal abutment, an appropriate amount of tooth removal must be complied with, and in order to comply with the appropriate amount of tooth removal, it is important for the user to maintain a constant angle of the handpiece during the procedure. Accordingly, when the user device 40 according to the twelfth embodiment of the present disclosure transmits the user's prescription execution information to the prescription system 130, the prescription system 130 may transmit feedback information on whether the user maintains a constant angle of the handpiece during the process of forming the abutment. If the user does not constantly keep the angle of the handpiece during the abutment forming process, then the notification unit 440 may generate vibration or a warning sound to aid the user in constantly keeping the angle of the handpiece.

In this way, the user device 40 according to the twelfth embodiment may provide the user (diagnostician) with sophistication and accuracy in treatment when treating a patient, and provide standards for treatment techniques that the user (diagnostician) has relied on based on his/her experience and know-how, and may also aid a trainee user (diagnostician) in building his or her skills.

In the above, embodiments in the method of providing a medical service for clinical diagnosis and prescription of the present disclosure and the system for the same have all been described.

Meanwhile, the present disclosure is not limited to the foregoing specific embodiments and application examples, it will be of course understood by those skilled in the art that various modifications may be made without departing from the gist of the present disclosure as defined in the following claims, and it is to be noted that those modifications should not be understood individually from the technical concept and prospect of the present disclosure.

1. A method of providing, by a teeth information processing system included in a treatment system, a medical service through processing teeth information and utilizing the processed information, the method comprising:
generating a dental data code for at least one tooth in a teeth image acquired by a computing device in the teeth information processing system,
wherein the dental data code comprises a coordinate code for identifying an intraoral location of at least each tooth.

2. The method of claim 1, wherein the dental data code further comprises:
an inspection code for distinguishing a type of teeth inspection item; and
an evaluation code indicating a condition according to an inspection result of teeth that have been inspected.

3. The method of claim 1, further comprising:
acquiring a photographed image of teeth, prior to generating the dental data code;
acquiring a teeth development image based on the photographed image; and
identifying individual tooth surfaces from the teeth development image.

4. A method of providing, by a diagnosis support system included in a treatment system, a medical service for diagnosis support to a certain person, the method comprising:
receiving an input from a diagnostician terminal to define a plurality of factors and a diagnosis support algorithm;
acquiring examination information, the examination information being information on the shape, location or condition of a patient's teeth, from an examination device;
executing the defined diagnosis support algorithm to generate diagnosis support data; and
providing the diagnosis support data to the diagnostician terminal.

5. The method of claim 4, wherein the executing of the diagnosis support algorithm comprises:
preprocessing the examination information;
standardizing the examination information; and
generating diagnosis support data based on the standardized examination information.

6. The method of claim 5, wherein the preprocessing of the examination information comprises:
analyzing the examination information;
removing the unnecessary data; and
determining whether the minimum examination information required to generate the diagnosis support data has been acquired.

7. A method of providing, by a prescription system included in a treatment system, a medical service for a prescription to a certain diagnostician or user, the method comprising:
acquiring teeth information related to the teeth of a certain subject person; and
performing calculation based on the teeth information, and generating prescription information corresponding to the subject person,
wherein the teeth information comprises at least one of the location information of the teeth and the condition information of the teeth.

8. The method of claim 7, further comprising:
subsequent to generating the prescription information, transmitting the generated prescription information to a certain device, the device comprising a dental care device.

9. The method of claim 8, further comprising:
subsequent to transmitting the generated prescription information to a certain device, receiving prescription execution information from the device,
wherein the prescription execution information comprises a value, an image, a video, or a code readable by the prescription system according to a user's prescription execution action.

10. The method of claim 9, further comprising:
subsequent to receiving prescription execution information from the device, performing calculation based on either one of the teeth information and the prescription information, and the prescription execution information to generate feedback information.
